(19) United States
(12) Patent Application Publication
(10) Pub. No.: US 2003/0134844 A1
(43) Pub. Date: Jul. 17, 2003

(54) Nicontinic acetylcholine receptor antagonists in the treatment of restless legs syndrome

(75) Inventor: Mario D. Saltarelli, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/224,055

(22) Filed: Aug. 20, 2002

Related U.S. Application Data
(60) Provisional application No. 60/334,810, filed on Oct. 31, 2001.

(51) Int. Cl. A61K 31/55; A61K 31/541; A61K 31/5377; A61K 31/496; A61K 31/473

(52) U.S. Cl. 514/217.07; 514/228.2; 514/232.8; 514/253.03; 514/290

(57) ABSTRACT

This invention relates to the use of nicotinic acetylcholine receptor agonists for the treatment of restless legs syndrome (RLS). The invention further relates to the use of a nicotinic acetylcholine receptor agonist in the manufacture of a medicament for the treatment of RLS. The present invention also relates to a pharmaceutical composition for the treatment of RLS containing a nicotinic acetylcholine receptor agonist.

### BACKGROUND OF THE INVENTION

This invention relates to the use of nicotinic acetylcholine receptor agonists for the treatment of restless legs syndrome (RLS). The invention also relates to the use of a nicotinic acetylcholine receptor agonist in the manufacture of a medicament for the treatment of RLS. The invention further relates to a pharmaceutical composition for the treatment of RLS containing a nicotinic acetylcholine receptor agonist.

Restless legs syndrome is a condition of unknown origin characterized by a bothersome, but usually not painful, sensation in one or both legs that causes an afflicted individual to experience an irresistible urge to move the legs. Occasionally, this condition occurs in the arms as well. Voluntary movement of the limb in which such a sensation is felt reportedly reduces or alleviates the intensity of the sensation. RLS most often affects its sufferers worst, or exclusively, when the afflicted individual is at rest or lying down in the evening or at night. Movement of the toes, feet or legs is typically observed in an afflicted individual when sitting or lying down, and has often been mischaracterized as fidgetiness or nervousness. A sufferer of RLS often may have difficulty falling and staying asleep with an estimated 80% of afflicted individuals having periodic limb movements throughout the night, sometimes as frequently as every 20 to 30 seconds, often causing partial arousal that disrupts sleep. The resulting chronic sleep deprivation and accompanying daytime fatigue often can cause mood swings in the afflicted individual and can have a debilitating effect on that individual's ability to work and function on a daily basis.

At present, the most prescribed treatment for RLS is a dopaminergic agent (often a dopamine-receptor agonist) like Mirapex (pramipexole), Permax (pergolide), and Requip (ropinirole), or a drug that adds dopamine to the system like Sinemet (carbidopa/levodopa). Of the dopaminergic agents, Sinemet has been used the longest, but has recently been found to cause the serious side effect of augmentation in the vast majority of patients who take it for the treatment of RLS. Other less used treatments for RLS are sedatives, which can relieve nighttime symptoms of RLS; pain relievers (including codeine, Darvon or Darvocet (propoxyphene), Dolophine (methadone), Percocet (oxycodone), Ultram (tramadol), and Vicodin (hydrocodone) for those with severe unrelenting symptoms of RLS; and anti-convulsants (including Gabapentin (Neurontin)) which are effective for some, but not all, patients with marked daytime symptoms, particularly people who have pain syndromes associated with their RLS.

Agonists of nicotinic acetylcholine receptors markedly increase the release of dopamine in the brain. As enhanced dopaminergic activity has been implicated in possible mechanisms of alleviation of RLS and dopaminergic agents have been somewhat effective in the treatment of RLS, agonists of the nicotinic acetylcholine specific receptors provide an alternative means to treat RLS avoiding some of the side effects associated with some known dopaminergic agents.

In particular, a number of compounds which bind to neuronal nicotinic receptor sites and are useful in modulating cholinergic function are referred to in International Patent Publication No. WO 01/62736, filed Feb. 8, 2001; International Patent Publication No. WO 99/35131, filed Nov. 13, 1998; International Patent Publication No. WO 99/55680, filed Apr. 8, 1999; International Patent Publication No. WO 98/18798, filed Oct. 15, 1997; U.S. Pat. No. 5,977,131, filed Mar. 31, 1998; U.S. Pat. No. 6,020,335, filed Nov. 4, 1997; and European Patent Publication No. EP 0 955 301 A2, filed Mar. 25, 1999. The foregoing applications are owned in common with the present application, and is incorporated herein by reference in their entirety.

### SUMMARY OF THE INVENTION

The present invention relates to a method of treating a mammal, including a human, for restless legs syndrome comprising administering to the mammal in need of such treatment an amount of a nicotinic acetylcholine receptor agonist effect in treating said syndrome.

The present invention further relates to a method of treating a mammal, including a human, for restless legs syndrome comprising administering to the mammal in need of such treatment an amount of a compound of formula I:

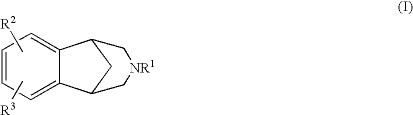

wherein

R¹ is hydrogen, (C₁-C₆)alkyl, unconjugated (C₃-C₆)alkenyl, benzyl, XC(═O)R¹³ or —CH₂CH₂—O—(C₁-C₄)alkyl;

R² and R³ are selected, independently, from hydrogen, (C₂-C₆)alkenyl, (C₂-C₆)alkynyl, hydroxy, nitro, amino, halo, cyano, —SO_{q}(C₁-C₆)alkyl wherein q is zero, one or two, (C₁-C₆)alkylamino—, [(C₁-C₆)alkyl]₂amino—, —CO₂R⁴, —CONR⁵R⁶, —SO₂NR⁷R⁸, —C(═O)R¹³, —XC(═O)R¹³, aryl-(C₀-C₃)alkyl— or aryl-(C₀-C₃)alkyl—O—, wherein said aryl is selected from phenyl and naphthyl, heteroaryl-(C₀-C₃)alkyl— or heteroaryl-(C₀-C₃)alkyl—O—, wherein said heteroaryl is selected from five to seven membered aromatic rings containing from one to four heteroatoms selected from oxygen, nitrogen and sulfur; X²(C₀-C₆)alkyl— and X²(C₁-C₆)alkoxy-(C₀-C₆)alkyl—, wherein X² is absent or X² is (C₁-C₆)alkylamino— or [(C₁-C₆)alkyl]₂amino—, and wherein the (C₀-C₆)alkyl— or (C₁-C₆)alkoxy-(C₀-C₆)alkyl— moieties of said X²(C₀-C₆)alkyl— or X²(C₁-C₆)alkoxy-(C₀-C₆)alkyl— contains at least one carbon atom, and wherein from one to three of the carbon atoms of said (C₀-C₆)alkyl— or (C₁-C₆)alkoxy-(C₀-C₆)alkyl— moieties may optionally be replaced by an oxygen, nitrogen or sulfur atom, with the proviso that any two such heteroatoms must be separated by at least two carbon atoms, and wherein any of the alkyl moieties of said (C₀-C₆)alkyl— or (C₁-C₆)alkoxy-(C₀-C₆)alkyl— groups may be optionally substituted with from two to seven fluorine atoms, and wherein one of the carbon atoms of each of the alkyl moieties of said aryl-(C₀-C₃)alkyl— and said heteroaryl-(C₀-C₃)alkyl— may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆)alkyl optionally substituted with from one to seven fluorine atoms, (C₁-C₆)alkoxy optionally substituted with from two to seven fluorine atoms, halo (e.g., chloro, fluoro, bromo or iodo), (C₂-C₆)alkenyl, (C₂-C₆)alkynyl, hydroxy, nitro, cyano, amino, (C₁-C₆)alkylamino—, [(C₁-C₆)alkyl]₂amino—, —CO₂R⁴, —CONR⁵R⁶, —SO₂NR⁷R⁸, —C(═O)R¹³ and —XC(═O)R¹³;

or R² and R³, together with the carbons to which they are attached, form a four to seven membered monocyclic, or a ten to fourteen membered bicyclic, carbocyclic ring that can be saturated or unsaturated, wherein from one to three of the non-fused carbon atoms of said monocyclic rings, and from one to five of the carbon atoms of said bicyclic rings that are not part of the benzo ring shown in formula I, may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆)alkyl— or (C₁-C₆)alkoxy-(C₀-C₆)alkyl—, wherein the total number of carbon atoms does not exceed six and wherein any of the alkyl moieties may optionally be substituted with from one to seven fluorine atoms; nitro, oxo, cyano, halo, (C₂-C₆)alkenyl, (C₂-C₆)alkynyl, hydroxy, amino, (C₁-C₆)alkylamino—, [(C₁-C₆)alkyl]₂amino—, —CO₂R⁴, —CONR⁵R⁶, —SO₂NR⁷R⁸, —C(═O)R¹³, and —XC(═O)R¹³;

each R⁴, R⁵, R⁶, R⁷, R⁸ and R¹³ is selected, independently, from hydrogen and (C₁-C₆) alkyl, or R⁵ and R⁶, or R⁷ and R⁸ together with the nitrogen to which they are attached, form a pyrrolidine, piperidine, morpholine, azetidine, piperazine, —N—(C₁-C₆)alkylpiperazine or thiomorpholine ring, or a thiomorpholine ring wherein the ring sulfur is replaced with a sulfoxide or sulfone; and

each X is, independently, (C₁-C₆)alkylene;

with the proviso that: (a) at least one of R¹, R² and R³ must be the other than hydrogen, and (b) when R² and R³ are hydrogen, R¹ cannot be hydrogen, (C₁-C₆)alkyl, or unconjugated (C₃-C₆)alkenyl, and pharmaceutically acceptable salts of such compounds.

Examples of possible heteroaryl groups within the definition of R² and R³ in formula I are the following: thienyl, oxazoyl, isoxazolyl, pyridyl, pyrimidyl, thiazolyl, tetrazolyl, isothiazolyl, triazolyl, imidazolyl, tetrazolyl, pyrrolyl and the following groups:

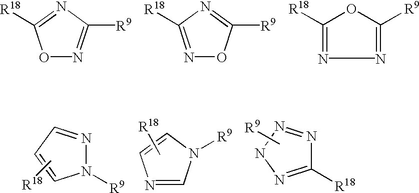

wherein one of R⁹ and R¹⁸ is hydrogen or (C₁-C₆)alkyl, and the other is a bond to the benzo ring of formula I.

Examples of compounds of the formula I used in the method of the invention are wherein R² and R³, together with the benzo ring of formula I, form a bicyclic ring system selected from the following:

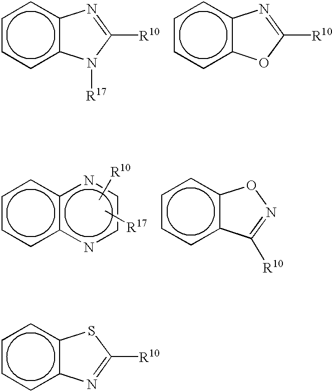

wherein R¹⁰ and R¹⁷ are selected, independently, from hydrogen, (C₁-C₆)alkyl; and (C₁-C₆)alkoxy-(C₀-C₆)alkyl— wherein the total number of carbon atoms does not exceed six and wherein any of the alkyl moieties may optionally be substituted with from one to seven fluorine atoms; nitro, cyano, halo, amino, (C₁-C₆)alkylamino—, [(C₁-C₆) alkyl]₂amino—, —CO₂R⁴, —CONR⁵R⁶, —SO₂NR⁷R⁸, —C(═O)R¹³, —XC(═O)R¹³, phenyl and monocyclic heteroaryl wherein said heteroaryl is defined as R² and R³ are defined in the definition of compounds of the formula I above;

Other embodiments compounds of the formula I in the method of the invention are wherein R² and R³, together with the benzo ring of formula I, form a bicyclic or tricyclic ring system selected from the following:

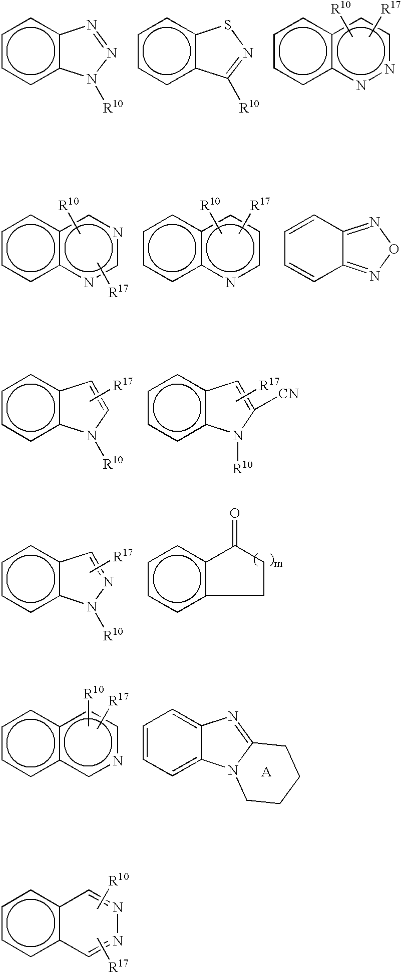

wherein R¹⁰ and R¹⁷ are defined as above, and m is zero, one or two, and wherein one of the carbon atoms of ring A can optionally be replaced with oxygen or N(C₁-C₆)alkyl.

Other embodiments of the compounds of the formula I in the methods of the invention are wherein neither R² nor R³ is attached to the benzo ring of formula I via an oxygen atom.

Other embodiments of this invention relate to compounds of the formula I, and their pharmaceutically acceptable salts, wherein R² and R³ do not, together with the benzo ring of formula I, form a bicyclic or tricyclic ring system.

Other embodiments of this invention relate to compounds of the formula I wherein one or both of R² and R³ are —C(═O)R¹³, wherein R¹³ is (C₁-C₆)alkyl. Further embodiments of this invention relate to compounds of the formula I wherein one or both of R² and R³ are —C(═O)R¹³, wherein R¹³ is (C₁-C₆)alkyl or (C₁-C₃)alkyl optionally substituted with from one to seven fluorine atoms. Other embodiments relate to compounds of the formula I wherein one of R² and R³ is CF₃, fluoro, cyano, (C₂-C₆)alkynyl or C₂F₅.

Examples of specific compounds of the formula I in the methods of the invention are the following compounds, which, in the instances where there is a center or centers of asymmetry in the molecule, may comprise a racemic mixture or the single enantiomer:

10-aza-tricyclo[6.3.1.0^{2,7}]dodeca-2(7),3,5-triene;

4-fluoro-10-aza-tricyclo[6.3.1.0^{2,7}]dodeca-2(7),3,5-triene;

4-methyl-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

3-trifluoromethyl-10-aza-tricyclo[6.3.1.0^{2,7}]dodeca-2(7),3,5-triene;

3-fluoro-10-aza-tricyclo[6.3.1.0^{2,7}]dodeca-2(7),3,5-triene;

4-nitro-10-azatricyclo[6.3.1.0^{2,7}]dodeca-2(7),3,5-triene;

4-amino-10-azatricyclo[6.3.1.0^{2,7}]dodeca-2(7),3,5-triene;

N¹-[10-azatricyclo[6.3.1.0^{2,7}]dodeca-2(7),3,5-trien-4-yl]-acetamide;

6-methyl-5-thia-7,13-diazatetracyclo[9.3.1.0^{2,10}.0^{4,8}]pentadeca-2(10),3,6,8-tetraene;

6-methyl-7-propyl-5,7,13-triazatetracyclo[9.3.1.0^{2,10}.0^{4,8}]-pentadeca-2(10),3,5,8-tetraene;

5,7,13-triazatetracyclo[9.3.10^{2,10}.0^{4,8}]-pentadeca-2(10),3,5,8-tetraene;

7-methyl-5,7,13-triazatetracyclo[9.3.10^{2,10}.0^{4,8}]-pentadeca-2(10),3,5,8-tetraene;

6-methyl-5,7,13-triazatetracyclo[9.3.1.0^{2,10}.0^{4,8}]-pentadeca-2(10),3,5,8-tetraene;

6,7-dimethyl-5,7,13-triazatetracyclo[9.3.1.0^{2,10}.0^{4,8}]-pentadeca-2(10),3,5,8-tetraene;

7-propyl-5,7,13-triazatetracyclo[9.3.1.0^{2,10}.0^{4,8}]-pentadeca-2(10),3,5,8-tetraene;

7-butyl-5,7,13-triazatetracyclo[9.3.1.0^{2,10}.0^{4,8}]-pentadeca-2(10),3,5,8-tetraene;

7-isobutyl-5,7,13-triazatetracyclo[9.3.1.0^{2,10}.0^{4,8}]-pentadeca-2(10),3,5,8-tetraene;

6-methyl-7-isobutyl-5,7,13-triazatetracyclo[9.3.1.0^{2,10}.0^{4,8}]-pentadeca-2(10),3,5,8-tetraene;

7-phenyl-5,7,13-triazatetracyclo[9.3.1.0^{2,10}.0^{4,8}]-pentadeca-2( 10),3,5,8-tetraene;

6-methyl-7-phenyl-5,7,13-triazatetracyclo[9.3.1.0^{2,10}.0^{4,8}]-pentadeca-2(10),3,5,8-tetraene;

6-methyl-7-neopentyl-5,7,13-triazatetracyclo[9.3.1.0^{2,10}.0^{4,8}]-pentadeca-2(10),3,5,8-tetraene;

6,7-dimethyl-5,8,14-triazatetracyclo[10.3.10^{2,11}.0^{4,9}]-hexadeca-2(11),3,5,7,9-pentaene;

5,8,14-triazatetracyclo[10.3.1.0^{2,11}.0^{4,9}]-hexadeca-2(11),3,5,7,9-pentaene;

14-methyl-5,8,14-triazatetracyclo[10.3.1.0^{2,11}.0^{4,9}]-hexadeca-2(11),3,5,7,9-pentaene;

5-oxa-7,13-diazatetracyclo[9.3.1.0^{2,10}.0^{4,8}]-pentadeca-2(10),3,6,8-tetraene;

6-methyl-5-oxa-7,13-diazatetracyclo[9.3.1.0^{2,10}.0^{4,8}]-pentadeca-2(10),3,6,8-tetraene;

2-fluoro-N-(4-hydroxy-10-aza-tricyclo[6.3.1.0^{2,7}]-dodeca-2(7),3,5-trien-5-yl)-benzamide;

4-chloro-10-azatricyclo[6.3.1.0^{2,7}]dodeca-2(7),3,5-triene;

10-azatricyclo[6.3.1.0^{2,7} ]dodeca-2(7),3,5-trien-4-yl cyanide;

3-(10-azatricyclo[6.3.1.0^{2,7}]dodeca-2(7),3,5-trien-4-yl)-5-methyl-1,2,4-oxadiazole;

1-(10-azatricyclo[6.3.1.0^{2,7}]dodeca-2(7),3,5-trien-4-yl)-1-ethanone;

10-azatricyclo[6.3.1.0^{2,7}]dodeca-2(7),3,5-trien-4-ol;

7-methyl-5-oxa-6,13-diazatetracyclo[9.3.1.0^{2,10}.0^{4,8}]pentadeca-2,4(8),6,9-tetraene;

4-(2-methyl-2H-pyrazol-3-yl)-10-aza-tricyclo[6.3.1.0^{2,7}]dodeca-2(7),3,5-triene;

4-(1-methyl-1H-pyrazol-3-yl)-10-aza-tricyclo[6.3.1.0^{2,7}]dodeca-2(7),3,5-triene;

4,5-dichloro-10-azatricyclo[6.3.1.0^{2,7}]dodeca-2(7),3,5-triene;

N⁴, N⁴-dimethyl-10-azatricyclo[6.3.1.0^{2,7}]-dodeca-2(7),3,5-triene-4-sulfonamide;

4-(1-pyrrolidinylsulfonyl)-10-azatricyclo[6.3.1.0^{2,7}]-dodeca-2(7),3,5-triene;

5,13-diazatetracyclo[9.3.1.0^{2,10}.0^{4,8}]-pentadeca-2,4(8),9-trien-6-one;

6-oxo-5-oxa-7,13-diazatetracyclo[9.3.1.0^{2,10}.0^{4,8}]-pentadeca-2(10),3,6,8-tetraene;

3-phenyl-10-aza-tricyclo[6.3.1.0^{2,7}]dodeca-2(7),3,5-triene;

3-hydroxy-10-aza-tricyclo[6.3.1.0^{2,7}]dodeca-2(7),3,5-triene;

4,5-difluoro-10-aza-tricyclo[6.3.1.0^{2,7}]dodeca-2(7),3,5-triene;

6-ethyl-5-oxa-7,13-diazatetracyclo[9.3.1.0^{2,10}.0^{4,8}]-pentadeca-2(10),3,6,8-tetraene;

6-isopropyl-5-oxa-7,13-diazatetracyclo[9.3.1.0^{2,10}.0^{4,8}]-pentadeca-2(10),3,6,8-tetraene;

6-benzyl-5-oxa-7,13-diazatetracyclo[9.3.1.0^{2,10}.0^{4,8}]-pentadeca-2(10),3,6,8-tetraene;

5,14-diazatetracyclo[10.3.1.0^{2,11}.0^{4,9}]hexadeca-2(11),3,5,7,9-pentaene;

6-methyl-5,14-diazatetracyclo[10.3.1.0^{2,11}.0^{4,9}]hexadeca-2(11),3,5,7,9-pentaene;

7-methyl-5,14-diazatetracyclo[10.3.1.0^{2,11}.0^{4,9}]hexadeca-2(11),3,5,7,9-pentaene;

7-ethyl-5,14-diazatetracyclo[10.3.1.0^{2,11}.0^{4,9}]hexadeca-2(11),3,5,7,9-pentaene;

8-methyl-5,14-diazatetracyclo[10.3.1.0^{2,11}.0^{4,9}]hexadeca-2(11),3,5,7,9-pentaene;

5,14-diazatetracyclo[10.3.1.0^{2,11}.0^{4,9}]hexadeca-2(11),3,7,9-tetraen-6-one;

6-chloro-5,14-diazatetracyclo[10.3.1.0^{2,11}.0^{4,9}]hexadeca-2(11),3,5,7,9-pentaene;

6-methoxy-5,14-diazatetracyclo[10.3.1.0^{2,11}.0^{4,9}]hexadeca-2(11),3,5,7,9-pentaene;

6-chloro-10-fluoro-5,14-diazatetracyclo[10.3.1.0^{2,11}.0^{4,9}]hexadeca-2(11),3,5,7,9-pentaene;

5,8,14-triazatetracyclo[10.3.1.0^{2,11}.0^{4,9}]hexadeca-2(11),3,7,9-tetraen-6-one;

and pharmaceutically acceptable salts and optical isomers thereof.

The present invention further relates to a method of treating a mammal, including a human, for restless legs syndrome comprising administering to the mammal in need of such treatment an compound of formula II

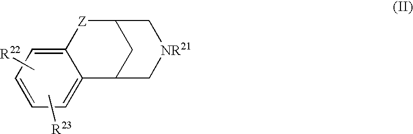

wherein Z is CH₂, C(═O) or CF₂;

R²¹ is hydrogen, (C₁-C₆)alkyl, unconjugated (C₃-C₆)alkenyl, benzyl, XC(═O)R¹³ or —CH₂CH₂—O—(C₁-C₄)alkyl;

R²² and R²³ are selected independently, from hydrogen, (C₂-C₆) alkenyl, (C₂-C₆) alkynyl, hydroxy, nitro, amino, halo, cyano, —SO_{q}(C₁-C₆)alkyl wherein q is zero, one or two, (C₁-C₆)alkylamino, [(C₁-C₆)alkyl]₂amino, CO₂R⁴, CONR⁵R⁶, SO₂NR⁷R⁸, C(═O)R¹³, XC(═O)R¹³, aryl-(C₀-C₃) alkyl or aryl-(C₀-C₃)alkyl-O— wherein said aryl is selected from phenyl and naphthyl, heteroaryl-(C₀-C₃)alkyl or heteroaryl-(C₀-C₃)alkyl-O—, wherein said heteroaryl is selected from five to seven membered aromatic rings containing from one to four heteroatoms selected from oxygen, nitrogen and sulfur, and X²(C₀-C₆)alkoxy-(CO-C₆)alkyl, wherein X² is absent or X² is (C₁-C₆)alkylamino or [(C₁-C₆)alkyl]₂amino, and wherein the (C₀-C₆)alkoxy-(CO-C₆)alkyl moiety of said X²(C₀-C₆)alkoxy-(C₀-C₆)alkyl contains at least one carbon atom, and wherein from one to three of the carbon atoms of said (C₀-C₆)alkoxy-(C₀-C₆)alkyl moiety may optionally be replaced by an oxygen, nitrogen or sulfur atom, with the proviso that any two such heteroatoms must be separated by at least two carbon atoms, and wherein any of the alkyl moieties of said (C₀-C₆)alkoxy-(C₀-C₆)alkyl may be optionally substituted with from two to seven fluorine atoms, and wherein one of the carbon atoms of each of the alkyl moieties of said aryl-(C₀-C₃)alkyl and said heteroaryl-(C₀-C₃)alkyl may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆) alkyl optionally substituted with from one to seven fluorine atoms, (C₁-C₆) alkoxy optionally substituted with from two to seven fluorine atoms, halo (e.g., chloro, fluoro, bromo or iodo), hydroxy, nitro, cyano, amino, (C₁-C₆) alkylamino and [(C₁-C₆) alkyl]₂ amino;

or R²² and R²³, together with the carbons to which they are attached, form a four to seven membered monocyclic, or a ten to fourteen membered bicyclic, carbocyclic ring that can be saturated or unsaturated, wherein from one to three of the nonfused carbon atoms of said monocyclic rings, and from one to five of the carbon atoms of said bicyclic rings that are not part of the benzo ring shown in formula II, may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆) alkoxy-(C₀-C₆)alkyl—, wherein the total number of carbon atoms does not exceed six and wherein any of the alkyl moieties may optionally be substituted with from one to seven fluorine atoms; nitro, oxo, cyano, halo, hydroxy, amino, (C₁-C₆)alkylamino, [(C₁-C₆)alkyl]₂amino, phenyl and monocyclic heteroaryl wherein said heteroaryl is defined as in the definition of R²² and R²³ above;

each R⁴, R⁵, R⁶, R⁷, R⁸, and X is as defined above;

with the proviso that: (a) at least one of R²¹, R²² and R²³ must be the other than hydrogen, (b) when R²² and R²³ are hydrogen, R²¹ cannot be methyl or hydrogen; and (c) no fluorine atom in any of the fluoro substituted alkyl or alkoxy moieties of R²² and R²³ can be attached to a carbon that is attached to a heteroatom;

and the pharmaceutically acceptable salts of such compounds.

Examples of heteroaryl groups that each of R²² and R²³ in the compounds of formula II in the method of the invention are the following: thienyl, oxazoyl, isoxazolyl, pyridyl, pyrimidyl, thiazolyl, tetrazolyl, isothiazolyl, triazolyl, imidazolyl, tetrazolyl, pyrroyl and the following groups:

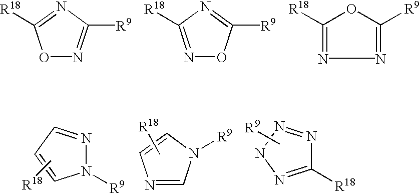

wherein R⁹ and R¹⁸ are as defined above.

Examples of compounds of the formula II in the methods of the invention are wherein R²² and R²³, together with the benzo ring of formula II, form a bicyclic ring system selected from the following:

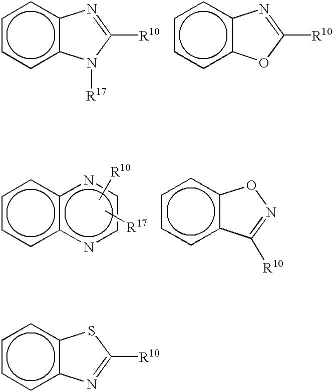

wherein R¹⁰ and R¹⁷ are as defined above;

Other embodiments of this invention relate to compounds of the formula II in the methods of the invention wherein R²² and R²³, together with the benzo ring of formula II, form a bicyclic or tricyclic ring system selected from the following:

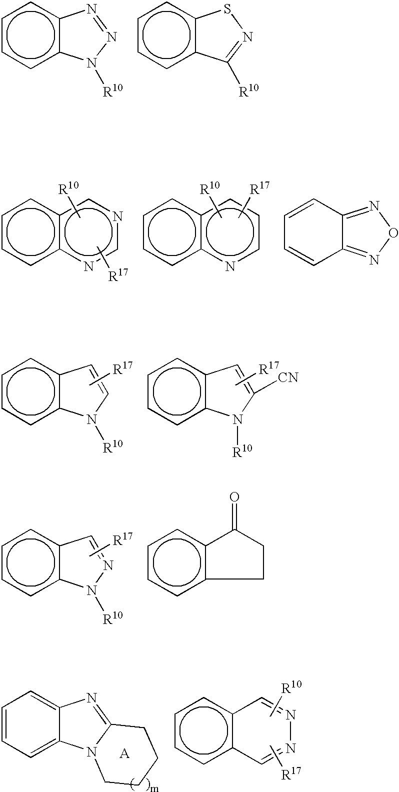

wherein m, R¹⁰ and R¹⁷ are as defined above and one of the carbon atoms of ring A can optionally be replaced with oxygen or —N(C₁-C₆)alkyl.

Other embodiments of this invention relate to compounds of the formula II in the methods of the invention wherein neither R²² nor R²³ is attached to the benzo ring of formula II via an oxygen atom.

Other embodiments of this invention relate to compounds of the formula II in the methods of the invention wherein R²¹ is not methyl.

Preferred embodiments of the invention relate to methods of treatment wherein the compounds of the formula II to be administered are selected from the group consisting of

5,6-difluoro-11-azatricyclo[7.3.1.0^{2,7}]trideca-2 4,6-triene;

11-benzyl-6-methoxy-11-aza-tricyclo[7.3.1.0^{2,7}]trideca-2(7),3,5-triene;

6-methoxy-11-aza-tricyclo[7.3.1.0^{2,7}]trideca-2(7),3,5-triene;

11-aza-tricyclo[7.3.1.0^{2,7}]trideca-2(7),3,5-trien-6-ol;

6-fluoro-11-aza-tricyclo[7.3.1.0^{2,7}]trideca-2(7),3,5-triene;

11-benzyl-5-methoxy-11-aza-tricyclo[7.3.1.0^{2,7}]trideca-2(7),3,5-triene;

11-benzyl-11-aza-tricyclo[7.3.1.0^{2,7}]trideca-2(7),3,5-trien-5-ol;

5-methoxy-11-aza-tricyclo[7.3.1.0^{2,7}]trideca-2(7),3,5-triene;

11-aza-tricyclo[7.3.1.0^{2,7}]trideca-2(7),3,5-trien-5-ol;

11-benzyl-5-difluoromethoxy-11-aza-tricyclo[7.3.1.0^{2,7}]trideca-2(7),3,5-triene;

5-difluoromethoxy-11-aza-tricyclo[7.3.1.0^{2,7}]trideca-2(7),3, 5-triene;

11-benzyl-5-ethoxy-11-aza-tricyclo[7.3.1.0^{2,7}]trideca-2(7),3,5-triene;

5-ethoxy-11-aza-tricyclo[7.3.1.0^{2,7}]trideca-2(7),3,5-triene;

5-isopropoxy-11-aza-tricyclo[7.3.1.0^{2,7}]trideca-2(7),3,5-triene;

11-benzyl-4-methoxy-11-aza-tricyclo[7.3.1.0^{2,7}]trideca-2(7),3,5-triene;

4-methoxy-11-aza-tricyclo[7.3.1.0^{2,7}]trideca-2(7),3,5-triene;

11-aza-tricyclo[7.3.1.0^{2,7}]trideca-2(7),3,5-trien-4-ol;

11-benzyl-11-aza-tricyclo[7.3.1.0^{2,7}]trideca-2(7),3,5-triene;

4-nitro-11-aza-tricyclo[7.3.1.0^{2,7}]trideca-2(7),3,5-triene;

5-nitro-11-aza-tricyclo[7.3.1.0^{2,7}]trideca-2(7),3,5-triene;

3-nitro-11-aza-tricyclo[7.3.1.0^{2,7}]trideca-2(7),3,5-triene;

11-benzyl-5-fluoro-11-aza-tricyclo[7.3.1.0^{2,7}]trideca-2(7),3,5-triene;

5-fluoro-11-aza-tricyclo[7.3.1.0^{2,7}]trideca-2(7),3,5-triene;

5,7-dioxa-14-azatetracyclo[10.3.1.0^{2,10}.0^{4,8}]hexadeca-2(10),3,8-triene;

11-benzyl-6-bromo-5-methoxy-11-aza-tricyclo[7.3.1.0^{2,7}]trideca-2(7),3,5-triene;

11-benzyl-6-hydroxy-5-methoxy-11-aza-tricyclo[7.3.1.0^{2,7}]trideca-2(7),3,5 -triene;

6-hydroxy-5-methoxy-11-aza-tricyclo[7.3.1.0^{2,7}]trideca-2(7),3,5 -triene;

trifluoromethanesulfonic acid-11-benzyl-11-aza-tricyclo[7.3.1.0^{2,7}]trideca-2(7),3,5-trien-5-yl ester;

5-(4-trifluoromethyl-phenyl)-11-aza-tricyclo[7.3.1.0^{2,7}]trideca-2(7),3,5-triene;

5-(4-methoxy-phenyl)-11-aza-tricyclo[7.3.1.0^{2,7}]trideca-2(7),3,5-triene;

11-aza-tricyclo[7.3.1.0^{2,7}]trideca-2(7),3,5-triene-5-carboxylic acid methyl ester;

2-(11-aza-tricyclo[7.3.1.0^{2,7}]trideca-2(7),3,5-trien-5-yl)-propan-2-ol;

5-pyridin-3-yl-11-aza-tricyclo[7.3.1.0^{2,7}]trideca-2(7),3,5-triene;

and pharmaceutically acceptable salts and optical isomers thereof.

The present invention also relates to a method of treating a mammal, including a human, for restless legs syndrome comprising administering to the mammal in need of such treatment an amount of a compound of formula III

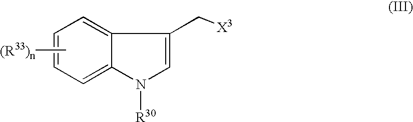

wherein X³ is:

a) —CH₂NR³¹R³²,

b)

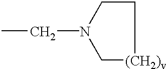

or

c)

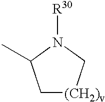

wherein R³⁰, R³¹, and R³² are independently selected from hydrogen and C₁-C₆ alkyl;

R³³ is selected from hydrogen, halogen and C₁-C₆ alkyl;

v is an integer from 0 to 4; and

n is an integer from 0 to 2; and pharmaceutically acceptable salts thereof.

Preferred compounds of formula III in the methods of the invention are:

[2-(6-chloro-1H-pyrrolo[2,3-b]pyridin-3-yl)ethyl]-dimethylamine;

[2-(6-chloro-1H-pyrrolo[2,3-b]pyridin-3-yl)ethyl]-methylamine;

3-pyrrolidin-2-ylmethyl-1H-pyrrolo[2,3-b]pyridine;

3-(1-methyl-pyrrolidin-2-ylmethyl)-1-H-pyrrolo[2,3-b]pyridine;

dimethyl-[2-(1H-pyrrolo[2,3-b]pyridin-3-yl)-ethyl]-amine;

methyl-[2-(1H-pyrrolo[2,3-b]pyridin-3-yl)-ethyl]-amine;

2-(1H-pyrrolo[2,3-b]pyridin-3-yl-ethylamine; and

3-(2-piperidin-1-yl-ethyl-1H-pyrrolo[2,3-b]pyridine.

The present invention further relates to a method of treating a mammal, including a human, for restless legs syndrome comprising administering to the mammal in need of such treatment an amount of a compound of formula IV:

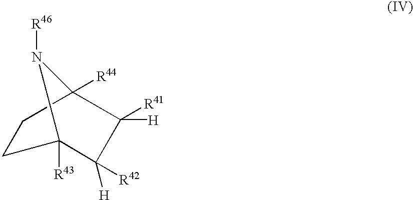

wherein R⁴¹, R⁴², R⁴³ and R⁴⁴ are selected, independently from hydrogen, —CO₂R⁴⁵, aryl and heteroaryl, wherein said aryl is selected from phenyl and naphthyl and said heteroaryl is selected from pyrazinyl, benzofuranyl, quinolyl, isoquinolyl, benzothienyl, isobenzofuryl, pyrazolyl, indolyl, isoindolyl, benzimidazolyl, purinyl, carbazolyl, 1,2,5-thiadiazolyl, quinazolinyl, pyridazinyl, pyrazinyl, cinnolinyl, phthalazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, and pyrazolopyrimidinyl oxazolyl, isoxazoyl, thiazolyl, isothiazolyl, furanyl, pyrazolyl, pyrrolyl, tetrazolyl, triazolyl, thienyl, imidazolyl, pyridinyl, and pyrimidinyl, and wherein said phenyl and said heteroaryl may optionally be substituted with from one to three substituents, and are preferably substituted with one or two substituents, independently selected form (C₁-C₆)alkyl optionally substituted with from one to seven (preferably with from zero to four) fluorine atoms, halo (i.e., chloro, fluoro, bromo or iodo), phenyl, benzyl, hydroxy, acetyl, amino, cyano, nitro, (C₁-C₆)alkoxy optionally substituted with from one to seven (preferably with from zero to four) fluorine atoms, (C₁-C₆)alkylamino and [(C₁-C₆)alkyl]₂amino;

R⁴⁵ is (C₁-C₆) alkyl, aryl, heteroaryl, (C₁-C₄)alkylene-aryl and (C₁-C₄)alkylene-heteroaryl, wherein said aryl and heteroaryl are defined as above, and wherein said (C₁-C₆)alkyl may optionally be substituted with from one to three substituents independently selected from halo, (C₁-C₆)alkyl, (C₁-C₆ )alkoxy, (C₁-C₄)alkoxy-(C₁-C₄)alkyl, amino, (C₁-C₆)alkylamino, and [(C₁-C₆)alkyl]₂amino; and

R⁴⁶ is hydrogen or (C₁-C₆)alkyl;

with the proviso that: (a) at least one of R⁴¹, R⁴², R⁴³, and R⁴⁴ must be aryl or heteroaryl; (b) when neither R⁴¹ nor R⁴² is hydrogen, R⁴¹ and R⁴² are in the “exo” configuration; (c) R⁴¹ and R⁴² can not both be —CO₂R⁴⁵; (d) if either R⁴³ or R⁴⁴ is —CO₂R⁴⁵ and R⁴⁵ is an alkyl or alkoxyalkyl group, then one of R⁴¹ and R⁴² must be aryl or heteroaryl; and (e) if either R⁴¹ or R⁴² is —CO₂R⁴⁵ and R⁴⁵ is an alkyl or alkoxyalkyl group, then one of R⁴³ and R⁴⁴ must be aryl or heteroaryl;

and the pharmaceutically acceptable salts of such compounds.

Preferred compounds of this invention include compounds of the formula IV in the methods of the invention wherein one of R⁴¹ and R⁴² is optionally substituted phenyl and the other is hydrogen, and wherein R⁴³ and R⁴⁴ are hydrogen.

More preferred compounds of the formula IV in the methods of the invention are wherein one of R⁴¹ and R⁴² is phenyl substituted with fluoro or nitro and the other is hydrogen, and wherein R⁴³ and R⁴⁴ are hydrogen.

More specific preferred embodiments of this invention are compounds of the formula IV in the methods of the invention wherein R⁴³ and R⁴⁴ are hydrogen and one R⁴¹ and R⁴² is hydrogen and the other is: (a) 3-fluorophenyl; (b) 4-nitrophenyl; or 3-fluoro-4-nitrophenyl.

Other embodiments of this invention relate to the following compounds of the formula IV and their pharmaceutically acceptable salts in the methods of the invention:

2β-(3,4-difluorophenyl)-7-aza-bicyclo[2.2.1]heptane;

2β-(3,5-dichlorobenzene)-7-aza-bicyclo[2.2.1]heptane;

2β-(4-nitrophenyl)-7-aza-bicyclo[2.2.1]heptane;

2β-(3-thiophene)-7-aza-bicyclo[2.2.1]heptane;

2β-(3-fluoro-4-chlorophenyl)-7-aza-bicyclo[2.2.1]heptane;

2β-(3-flourophenyl)-7-aza-bicyclo[2.2.1]heptane;

2β-(3-hydroxyphenyl)-7-aza-bicyclo[2.2.1]heptane;

2β-(3-acetophenone)-7-aza-bicyclo[2.2.1]heptane;

2β-(4-trifluoromethylphenyl)-7-aza-bicyclo[2.2.1]heptane;

2β-(3-fluoro-4-methylphenyl)-7-aza-bicyclo[2.2.1]heptane;

2β-(3-chlorophenyl)-7-aza-bicyclo[2.2.1]heptane;

2β-(n-benzyl-5-pyridonyl)-7-aza-bicyclo[2.2.1]heptane;

2β-(n-methyl-5-pyridonyl)-7-aza-bicyclo[2.2.1]heptane;

2β-(3-fluoro-5-nitrophenyl)-7-aza-bicyclo[2.2.1]heptane;

2β-(4-aminophenyl)-7-aza-bicyclo[2.2.1]heptane;

2β-(3-fluoro-4-trifluoromethyl-phenyl)-7-aza-bicyclo[2.2.1]heptane;

2β-(4-chlorophenyl)-7-aza-bicyclo[2.2.1]heptane;

2β-(3,4-methylenedioxyphenyl)-7-aza-bicyclo[2.2.1]heptane;

2β-(2-chloro-6-methyl-5-pyridinyl)-7-aza-bicyclo[2.2.1]heptane;

2β-(4-cyanophenyl)-7-aza-bicyclo[2.2.1]heptane;

2β-(3-fluoro-4-nitro-phenyl)-7-aza-bicyclo[2.2.1]heptane;

2β-(4-amido-phenyl)-7-aza-bicyclo[2.2.1]heptane;

2β-(3-fluoro-4-amino-phenyl)-7-aza-bicyclo[2.2.1]heptane;

2β-(4-sulfonamido-phenyl)-7-aza-bicyclo[2.2.1]heptane;

2β-(3-methyl-5-isoxzazole)-7-aza-bicyclo[2.2.1]heptane;

2β-(3-methyl-5-isoxzazole)-7-aza-bicyclo[2.2.1]heptane, N-methyl;

2β-(3-methyl-5-isoxzazole)-7-aza-bicyclo[2.2.1]heptane, N-acetyl;

2b-(3,4-difluorophenyl)-7-azabicyclo[2.2.1]heptane;

4-(7-aza-bicyclo[2.2.1]hept-2-yl)-benzamidine;

2-(4-methanesulfonyl-phenyl)-7-aza-bicyclo[2.2.1]heptane;

4-(7-aza-bicyclo[2.2.1]hept-2-yl)-phenol;

2-(4-methylsulfanyl-phenyl)-7-aza-bicyclo[2.2.1]heptane;

4-(7-aza-bicyclo[2.2.1]hept-2-yl)-benzoic acid methyl ester;

4-(7-aza-bicyclo[2.2.1]hept-2-yl)-benzoic acid;

2-(3-fluoro-4-tetrazol-1-yl-phenyl)-7-aza-bicyclo[2.2.1]heptane;

2-(4-nitro-3-trifluoromethyl-phenyl)-7-aza-bicyclo[2.2.1]heptane;

2-[3-fluoro-4-(5-trifluoromethyl-tetrazol-1-yl)-phenyl]-7-aza-bicyclo[2.2.1]heptane;

2-(3-chloro-4-nitro-phenyl)-7-aza-bicyclo[2.2.1]heptane;

2-(4-tetrazol-1-yl-phenyl)-7-aza-bicyclo[2.2.1]heptane;

2-(6-methoxy-pyridin-2-yl)-7-aza-bicyclo[2.2.1]heptane;

2-(4-methanesulfinyl-phenyl)-7-aza-bicyclo[2.2.1]heptane;

2-(4-bromo-3-fluoro-phenyl)-7-aza-bicyclo[2.2.1]heptane;

2-(4-cyano-3-fluoro-phenyl)-7-aza-bicyclo[2.2.1]heptane;

2-(3,4,5-trifluoro-phenyl)-7-aza-bicyclo[2.2.1]heptane;

2-(3,4,5-trimethoxy-phenyl)-7-aza-bicyclo[2.2.1]heptane;

2-(5-nitro-furan-2-yl)-7-aza-bicyclo[2.2.1]heptane;

5-(7-aza-bicyclo[2.2.1]hept-2-yl)-3-methyl-benzo[d]isoxazole;

6-(7-aza-bicyclo[2.2.1]hept-2-yl)-3-methyl-benzo[d]isoxazole;

6-(7-aza-bicyclo[2.2.1]hept-2-yl)-1,4-dihydro-quinoxaline-2,3-dione;

6-(7-aza-bicyclo[2.2.1]hept-2-yl)-quinoxaline; and

1-[4-(7-aza-bicyclo[2.2.1]hept-2-yl)-2-fluoro-phenyl]-ethanone

and pharmaceutically acceptable salts and optical isomers thereof.

The present invention further relates to a method of treating a mammal, including a human, for restless legs syndrome comprising administering to the mammal in need of such treatment an amount of a compound of formula V

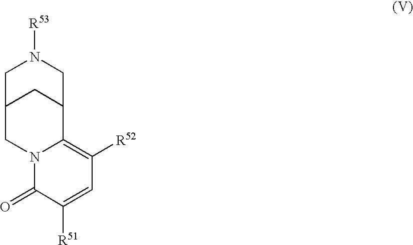

its enantiomers, diastereomers and stereoisomers, and their pharmaceutically acceptable salts and prodrugs,

wherein R⁵¹ and R⁵² are each independently selected from

a) H; halo; CF₃; hydroxy; (C₁-C₆)alkoxy; CH₂OH; —C(O)R⁵⁴, wherein R⁵⁴ is H, (C₁-C₆)alkyl, (C₆-C₁₀)aryl or benzyl (including substituted alkyl, aryl or benzyl); C≡N; C≡CR⁵⁵, wherein R⁵⁵ is H, (C₁-C₆)alkyl, (C₆-C₁₀)aryl (including substituted alkyl or aryl); —S(O)ₚR⁵⁵, wherein R⁵⁵ is H, (C₁-C₆)alkyl, or (C₆-C₁₀)aryl (including substituted alkyl or aryl) and p is 0, 1, or 2; (C₁-C₆)alkyl; (C₁-C₆)alkenyl; H₂N; di-((C₁-C₆)alkyl)amino; mono(C₁-C₆)alkyl-amino; (C₆-C₁₀)aryl-amino; (C₃-C₈)cycloalkyl-amino; heteroaryl-amino; cycloheteroalkyl-amino; and CON(R⁵⁵)₂ wherein each R⁵⁵ is selected from hydrogen, (C₁-C₆)alkyl and (C₆-C₁₀)aryl; and

b) CO₂R⁵⁶ wherein R⁵⁶ is selected from H, (C₁-C₆)alkyl, phenyl and benzyl; and

c) optionally benzene-fused (C₆-C₁₀)aryl, optionally benzene-fused (C₃-C₈)cycloalkyl, optionally benzene-fused heteroaryl and optionally benzene-fused cycloheteroalkyl, wherein said heteroaryl group contains five to ten atoms comprising one to four heteroatoms, said cycloheteroalkyl contains 4 to 8 atoms comprising one or two heteroatoms selected from N, S and O;

and wherein any of the alkyl, alkenyl, aryl, cycloalkyl, cycloheteroalkyl and heteroaryl groups in a), b) and c) are optionally substituted with one or more substituents selected from halogen, (C₁-C₆)alkyl, (C₆-C₁₀)aryl, hydroxy, hydroxymethyl, CHO and CO₂R⁵⁶ wherein R⁵⁶ is as described above; and

R⁵³ is selected from H, optionally substituted benzyl and methyl;

with the provisos that R⁵¹ and R⁵² are not both hydrogen and when R⁵³ is H, and that R⁵¹ and R⁵² when selected from H, Br and Cl are not be the same.

Preferred compounds of formula V in the methods of the invention are those wherein R⁵³ is selected from H, benzyl or methyl and R⁵¹ and R⁵² are each independently selected from H, halo, (C₁-C₆)alkyl, cyano, (C₆-C₁₀)aryl, (C₅-C₉)heteroaryl, (C₁-C₆)alkenyl, (C₂-C₆)alkynyl-R⁵⁵ and —C(O)R⁵⁵ wherein R⁵⁵ is H, (C₁-C₆) alkyl, (C₆-C₁₀)aryl and (C₅-C₉)heteroaryl and amino and mono and di-substituted amino; with the provisos that when R⁵³ is H then R⁵¹ and R⁵² are not both H, Br and Cl and when R⁵³ is benzyl or methyl then R⁵¹ and R⁵² are not hydrogen.

More preferred compounds of formula V in the methods of the invention are those wherein R⁵¹ and R⁵² are each independently selected from H, ethyl, methyl, phenyl, vinyl, fluoro, bromo, chloro, isopropyl, tert-butyl, trifluoromethyl, acetyl, propanoyl, 2,2-dimethylpropanoyl, 2-methylpropanoyl, butanoyl, pentanoyl, cyano, di-[(C₁-C₆)alkyl]amino, (C₁-C₆)monoalkylamino, (C₆-C₁₀)arylamino, (C₃-C₈)cycloalkylamino, heteroarylamino, cycloheteroalkyamino and CON(R⁵⁵)₂ wherein each R⁵⁵ is selected from hydrogen, (C₁-C₆)alkyl and (C₆-C₁₀)aryl; (C₆-C₁₀)aryl and (C₅-C₉)heteroaryl wherein the aryl and heteroaryl groups are optionally substituted with one or more substituents selected from halogen, (C₁-C₆)alkyl, (C₆-C₁₀)aryl, hydroxy, hydroxymethyl, CHO and CO₂R⁵⁶.

More preferred compounds of formula V in the methods of the invention are those wherein R⁵³ is selected from optionally substituted benzyl or (C₁-C₆)alkyl, wherein the substituents are described above and R⁵¹ and R⁵² are each independently selected from hydrogen, halo, cyano, optionally substituted (C₁-C₆)alkyl, (C₁-C₆)alkenyl, amino, di-[(C₁-C₆)alkyl]amino, (C₁-C₆)monoalkylamino, (C₆-C₁₀)arylamino, (C₃-C₈)cycloalkylamino, heteroarylamino, cycloheteroalkyamino and CON(R⁵⁵)₂ wherein each R⁵⁵ is selected from hydrogen, (C₁-C₆)alkyl and (C₆-C₁₀)aryl; —C(O)R⁵⁵ wherein R⁵⁵ is H, (C₁-C₆)alkyl, or (C₅-C₁₀)aryl; (C₆-C₁₀)aryl or (C₅-C₉)heteroaryl wherein the substituents are described above.

More particularly, the invention relates to compounds of the formula V in the methods of the invention wherein R⁵¹ and R⁵² are each independently selected from hydrogen isopropyl, tert-butyl, trifluoromethyl, acetyl, propanoyl, 2,2-dimethyipropanoyl, 2-methylpropanoyl, butanoyl, pentanoyl, cyano, 2,4-difluorophenyl, 2-fluorophenyl, 2- and 3-thienyl, dimethylamino and R⁵³ is selected from hydrogen, benzyl, methyl and R⁵¹ and R⁵² are each independently selected from hydrogen, bromo, chloro, ethyl, methyl, fluoro, vinyl and phenyl.

Most preferred compounds of the formula V in the methods of the invention are selected from:

9-bromo-1,2,3,4,5,6-hexahydro-1,5-methano-pyrido[1,2-a][1,5]diazocin-8-one;

11-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

11-chloro-1,2,3,4,5,6-hexahydro-1,5-methano-pyrido[1,2-a][1,5]diazocin-8-one;

9-flouro-1,2,3,4,5,6-hexahydro-1,5-methano-pyrido[1,2-a][1,5]diazocin-8-one;

11-flouro-1,2,3,4,5,6-hexahydro-1,5-methano-pyrido[1,2-a][1,5]diazocin-8-one;

9,11-diflouro-1,2,3,4,5,6-hexahydro-1,5-methano-pyrido[1,2-a][1,5]diazocin-8-one;

9-ethyl-1,2,3,4,5,6-hexahydro-1,5-methano-pyrido[1,2-a][1,5]diazocin-8-one;

11-ethyl-1,2,3,4,5,6-hexahydro-1,5-methano-pyrido[1,2-a][1,5]diazocin-8-one;

9,11-diethyl-1,2,3,4,5,6-hexahydro-1,5-methano-pyrido[1,2-a][1,5]diazocin-8-one;

9-methyl-1,2,3,4,5,6-hexahydro-1,5-methano-pyrido[1,2-a][1,5]diazocin-8-one;

11-methyl-1,2,3,4,5,6-hexahydro-1,5-methano-pyrido[1,2-a][1,5]diazocin-8-one;

9,11-dimethyl-1,2,3,4,5,6-hexahydro-1,5-methano-pyrido[1,2-a][1,5]diazocin-8-one;

9-phenyl-1,2,3,4,5,6-hexahydro-1,5-methano-pyrido[1,2-a][1,5]diazocin-8-one;

11-phenyl-1,2,3,4,5,6-hexahydro-1,5-methano-pyrido[1,2-a][1,5]diazocin-8-one;

9,11-diphenyl-1,2,3,4,5,6-hexahydro-1,5-methano-pyrido[1,2-a][1,5]diazocin-8-one ;

9-vinyl-1,2,3,4,5,6-hexahydro-1,5-methano-pyrido[1,2-a][1,5]diazocin-8-one;

11-vinyl-1,2,3,4,5,6-hexahydro-1,5-methano-pyrido[1,2-a][1,5]diazocin-8-one;

9,11-divinyl-1,2,3,4,5,6-hexahydro-1,5-methano-pyrido[1,2-a][1,5]diazocin-8-one;

9-bromo-3-methyl-1,2,3,4,5,6-hexahydro-1,5-methano-pyrido[1,2-a][1,5]diazocin-8-one;

3-benzyl-9-bromo-1,2,3,4,5,6-hexahydro-1,5-methano-pyrido[1,2-a][1,5]diazocin-8-one; and

3-benzyl-9-chloro-1,2,3,4,5,6-hexahydro-1,5-methano-pyrido[1,2-a][1,5]diazocin-8-one

and pharmaceutically acceptable salts and optical isomers thereof.

The present invention further relates to a method of treating a mammal, including a human, for restless legs syndrome comprising administering to the mammal in need of such treatment a compound of formula VI:

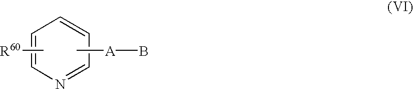

and their pharmaceutically acceptable acid addition salts and prodrugs,

wherein A is —CH(R⁶¹)— and R⁶¹ is hydrogen or optionally substituted (C₁-C₆)alkyl wherein the substituents comprise one or more groups individually selected from hydroxy, (C₁-C₆)alkoxy, oxo, (C₂-C₆)alkanoyl and NR⁶²R⁶³; and

B is a group of the formula

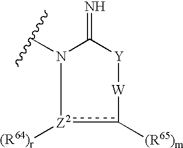

wherein Y—W is CH₂, NH, O, S, CH₂CH₂, CH═CH, N═CH, NH—CH₂, OCH₂ or SCH₂;

the dotted line represents an optional bond;

Z² is C, N, O or S;

m is 1 or 2;

r is 0, 1 or 2 with the proviso that r is 0 when Z² is O or S, r is 1 when Z² is N and r is 2 when Z² is C;

each R⁶⁴ and R⁶⁵ is independently selected from hydrogen, optionally substituted (C₁-C₆)alkyl, optionally substituted (C₁-C₆)alkoxy and optionally substituted (C₂-C₆)alkanoyl, wherein the substituents on the alkyl or alkanoyl groups are selected from hydroxy, (C₁-C₆)alkoxy, oxo, (C₂-C₆)alkanoyl and NR⁶²R⁶³, or R⁶⁴ and R⁶⁵ together with the carbon atoms to which they are attached form an optionally substituted six membered heteroaromatic ring containing at least one heteroatom selected from N, S and O and Z² is C wherein said substituents are selected from optionally substituted (C₁-C₆)alkyl or optionally substituted (C₁-C₆)alkoxy wherein said substituents are selected from (C₁-C₆)alkyl, optionally substituted (C₁-C₆)alkoxy and optionally substituted (C₂-C₆)alkanoyl or R⁶⁴ and one of R⁶⁵ together form a bond with the proviso that R⁶⁴ and R⁶⁵ cannot form a bond when Z² is O or S;

R⁶⁰ is hydrogen or halo; and

R⁶² and R⁶³ are each independently selected from hydrogen and optionally substituted (C₁-C₆)alkyl wherein said substituents are selected from (C₁-C₆)alkyl and halo;

with the provisos that when —B—A is attached to the 3-position of the pyridine ring and R⁶¹ is hydrogen and

a) R⁶⁰ is 6-chloro and

i) Z² is C, the dotted line represents a bond, m and r are both 1, R⁶⁴ and R⁶⁵ are both hydrogen, then W—Y is not selected from CH═CH, S, CH₂, NH, CH═N, OCH₂ or SCH₂;

ii) Z² is nitrogen, the dotted line represents a bond, r is 0 and m is 1 then R⁶⁵ is not CF₃; or

iii) Z² is C, the dotted line represents a bond, m and r are both 2, and each R⁶⁴ and R⁶⁵ is hydrogen, then W—Y is not S; or

b) R⁶⁰ is hydrogen, 6-bromo or 6-fluoro and Z² is carbon, the dotted line represents a bond, m and r are both 1, R⁶⁴ and R⁶⁵ are both hydrogen, then W—Y is not sulfur.

Preferred compounds of the formula VI in the methods of the invention are those wherein Z² is N, m is 1 or 2, W—Y is S or CH═CH, R⁶⁰ is halo or H, R⁶⁵ is (C₁-C₆)alkyl or halo, and the dotted line is a bond.

Other preferred compounds of the formula VI in the methods of the invention are those wherein Z² is C, R⁶¹ is (C₁-C₆)alkyl or hydrogen, m is 1, W—Y is S or CH═CH, the dotted line is a bond, R⁶⁴ and R⁶⁵ are each hydrogen or (C₁-C₆)alkyl, or the portion of B corresponding to

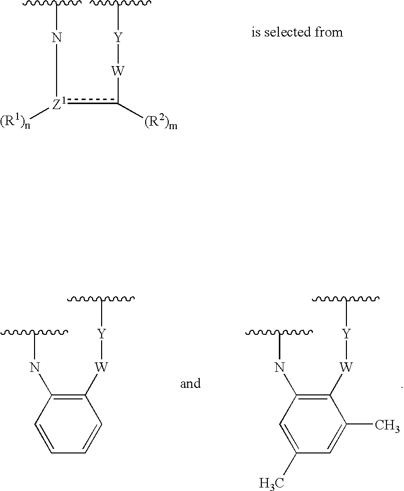

is selected from

and

Most preferred compounds of the formula VI in the methods of the invention are selected from the group comprising

3-(6-chloro-pyridin-3-ylmethyl)-3H-[1,3,4]thiadiazol-2-ylideneamine;

5-methyl-3-pyridin-3-ylmethyl-3H-thiazol-2-ylideneamine;

3-(6-chloro-pyridin-3-ylmethyl)-5-methyl-3H-[1,3,4]thiadiozol-2-ylideneamine;

6-chloro-2-(6-chloro-pyridin-3-ylmethyl)-2H-pyridazin-3-ylideneamine;

3-(6-chloro-pyridin-3-ylmethyl)-3H-benzothiazol-2-ylideneamine;

3-pyridin-3-ylmethyl-3H-[1,3,4]thiadiazol-2-ylideneamine;

3-[1-(6-chloro-pyridin-3-yl)-ethyl]-3H-thiazol-2-ylideneamine;

3-[1-(6-chloro-pyridin-3-yl)-ethyl]-3H-[1,3,4]thiadiazol-2-ylideneamine;

3-[1-(6-chloro-pyridin-3-ylmethyl)-thiazolidin-2-ylideneamine;

3-pyridin-3-ylmethyl-thiazolidin-2-ylideneamine;

5,7-dimethyl-1-pyridin-3-ylmethyl-3H-[1,8]naphthyridin-2-ylidene;

6-chloro-2-pyridin-3-ylmethyl-2H-pyridazin-3-ylideneamine; and

5-methyl-3-pyridin-3-ylmethyl-3H-[1,3,4]thiadiazol-2-ylideneamine

and pharmaceutically acceptable salts and optical isomers thereof.

The compounds of the formulae I, II, III, IV, V and VI may have optical centers and therefore may occur in different enantiomeric configurations. The invention includes all enantiomers, diastereomers, and other stereoisomers of such compounds of the formulae I, II, III, IV, V and VI as well as racemic and other mixtures thereof.

Preferably, the amount of the compounds of the formulae I, II, III, IV, V and VI administered in the methods of the invention are that which is effective in treating restless legs syndrome.

Unless otherwise indicated, the term “halo”, as used herein, includes fluoro, chloro, bromo and iodo.

Unless otherwise indicated, the term “alkyl”, as used herein, includes straight chain moieties, and where the number of carbon atoms suffices, branched and cyclic moieties.

The term “alkoxy”, as used herein, means “—O—alkyl” or “alkyl—O—”, wherein “alkyl” is defined as above.

The term “alkylene, as used herein, means an alkyl radical having two available bonding sites (i.e., —alkyl—), wherein “alkyl” is defined as above.

In the above compounds, “aryl” includes, without limitation, optionally substituted phenyl and naphthyl, “cycloalkyl” includes, without limitation, optionally substituted cyclopentyl and cyclohexyl, and said cycloalkyl group may also be unsaturated, and “heteroaryl” includes, without limitation, thienyl, furyl, pyrano, pyrrolo, imidazolyl, oxazolyl, thiazolyl, tetrazolyl, triazolyl, pyrazinyl and pyridyl, and said “cycloheteroalkyl” includes, without limitation, pyrrolidinyl, piperidinyl, tetrahydrofuryl and tetrahydropyrano.

Unless otherwise indicated, the term “one or more substituents”, as used herein, refers to from one to the maximum number of substituents possible based on the number of available bonding sites.

The term “nicotinic acetylcholine receptor agonist” refers to and encompasses full agonists of and partial agonists of nicotinic acetylcholine receptors.

The term “treatment”, as used herein, refers to reversing, alleviating, inhibiting the progress of, or preventing the disorder or condition to which such term applies, or one or more symptoms of such condition or disorder. The term “treatment”, as used herein, refers to the act of treating, as “treating” is defined immediately above.

The present invention also relates to all radiolabeled forms of the compounds of the formulae I, II, III, IV, V and VI. Preferred radiolabeled compounds of the formulae I, II, III, IV, V and VI are those wherein the radiolabels are selected from as ³H, ¹¹C, ¹⁴C, ¹⁸F, ¹²³I and ¹²⁵I. Such radiolabeled compounds are useful as research and diagnostic tools in metabolism studies, such as pharmacokinetics studies, etc., and in binding assays in both animals and man.

This invention also relates to the pharmaceutically acceptable acid addition salts of the compounds of the formulae I, II, III, IV, V and VI. Examples of pharmaceutically acceptable acid addition salts of the compounds of the formulae I, II, III, IV, V and VI are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, malic acid, di-p-toluoyl tartaric acid, and mandelic acid, as well salts formed from other acids known to those of skill in the art to form pharmaceutically acceptable acid addition salts to basic compounds. Other possible acid addition salts are, e.g., salts containing pharmaceutically acceptable anions, such as the hydroiodide, nitrate, sulfate or bisulfate, phosphate or acid phosphate, acetate, lactate, gluconate, saccharate, benzoate, methanesulfonate, ethanesulfonate, benzenesulfonate, and pamoate (i.e., 1.1′-methylene-bis-(2-hydroxy-3-naphthoate) salts).

This invention further relates to the use of nicotinic acetylcholine receptor agonists in the manufacture of a medicament for the treatment of restless legs syndrome (RLS). This invention further relates to the use of nicotinic acetylcholine receptor agonists selected from compounds of formulae I, II, III, IV, V and VI or a pharmaceutically acceptable salt thereof in the manufacture of a medicament for the treatment of restless legs syndrome (RLS). The present invention further relates to a pharmaceutical composition for the treatment of restless legs syndrome (RLS) comprising a compound selected from compounds of formulae I, II, III, IV, V and VI or a pharmaceutically acceptable salt thereof and a pharmaceutically acceptable carrier.

### DETAILED DESCRIPTION OF THE INVENTION

The present invention is drawn to the use of compounds which bind to neuronal nicotinic receptor sites and are useful in modulating cholinergic function for the treatment of restless legs syndrome. In particular, a number of compounds useful in the present invention are referred to in International Patent Publication No. WO 01/62736, filed Feb. 8, 2001 (compounds of formula I); International Patent Publication No. WO 99/35131, filed Nov. 13, 1998 (compounds of formula I); International Patent Publication No. WO 99/55680, filed Apr. 8, 1999 (compounds of formula II); U.S. Pat. No. 5,977,131, filed Mar. 31, 1998 (compounds of formula III); European Patent Publication No. EP 0 955 301 A2, filed Mar. 25, 1999 (compounds of formula IV); International Patent Publication No. WO 98/18798, filed Oct. 15, 1997 (compounds of formula V); and U.S. Pat. No. 6,020,335, filed Nov. 4, 1997 (compounds of formula VI).

The compounds of the formulae I, II, III, IV, V and VI and their pharmaceutically acceptable salts (hereafter “the active compounds”) can be administered via either the oral, transdermal (e.g., through the use of a patch), intranasal, sublingual, rectal, parenteral or topical routes. Transdermal and oral administration are preferred. These compounds are, most desirably, administered in dosages ranging from about 0.1 mg up to about 1500 mg per day, preferably from about 0.1 to about 300 mg per day, more preferably from about 0.1 to about 3 mg per day in single or divided doses, although variations will necessarily occur depending upon the particular compound used, the weight and condition of the subject being treated and the particular route of administration chosen. However, a dosage level that is in the range of about 0.001 mg to about 10 mg per kg of body weight per day is most desirably employed. Variations may nevertheless occur depending upon the weight and condition of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active compounds can be administered alone or in combination with pharmaceutically acceptable carriers or diluents by any of the several routes previously indicated. More particularly, the active compounds can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of a similar type may also be employed as fillers in gelatin capsules; preferred materials in this connection also include lactose or milk sugar, as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active compound in either sesame or peanut oil or in aqueous propylene glycol can be employed. The aqueous solutions should be suitably buffered (preferably pH greater than 8), if necessary, and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

It is also possible to administer the active compounds topically and this can be done by way of creams, a patch, jellies, gels, pastes, ointments and the like, in accordance with standard pharmaceutical practice.

### Biological Assay

The effectiveness of the active compounds in suppressing nicotine binding to specific receptor sites is determined by the following procedure which is a modification of the methods of Lippiello, P. M. and Fernandes, K. G. (in “The Binding of L-[³H]Nicotine To A Single Class of High-Affinity Sites in Rat Brain Membranes”, Molecular Pharm., 29: 448-54 (1986)) and Anderson, D. J. and Arneric, S. P. (in “Nicotinic Receptor Binding of ³H-Cytisine, ³H-Nicotine and ³H-Methylcarmbamylcholine In Rat Brain”, European J. Pharm., 253: 261-67 (1994)).

### Procedure

Male Sprague-Dawley rats (200-300 g) from Charles River were housed in groups in hanging stainless steel wire cages and were maintained on a 12 hour light/dark cycle (7 a.m.-7 p.m. light period). They received standard Purina Rat Chow and water ad libitum.

The rats were killed by decapitation. Brains were removed immediately following decapitation. Membranes were prepared from brain tissue according to the methods of Lippiello and Fernandez (Molecular Pharm., 29: 448-454 (1986) with some modifications. Whole brains were removed, rinsed with ice-cold buffer, and homogenized at 0° in 10 volumes of buffer (w/v) using a Brinkmann Polytron™, setting 6, for 30 seconds. The buffer consisted of 50 mM Tris HCl at a pH of 7.5 at room temperature. The homogenate was sedimented by centrifugation (10 minutes; 50,000× g; 0 to 4° C. The supernatant was poured off and the membranes were gently resuspended with the Polytron and centrifuged again (10 minutes; 50,000× g; 0 to 4° C. After the second centrifugation, the membranes were resuspended in assay buffer at a concentration of 1.0 g/100 mL. The composition of the standard assay buffer was 50 mM Tris HCl, 120 mM NaCl, 5 mM KCl, 2 mM MgCl₂, 2 mM CaCl₂ and has a pH of 7.4 at room temperature.

Routine assays were performed in borosilicate glass test tubes. The assay mixture typically consisted of 0.9 mg of membrane protein in a final incubation volume of 1.0 mL. Three sets of tubes were prepared wherein the tubes in each set contained 50 μL of vehicle, blank, or test compound solution, respectively. To each tube was added 200 μL of [³H]-nicotine in assay buffer followed by 750 μL of the membrane suspension. The final concentration of nicotine in each tube was 0.9 nM. The final concentration of cytisine in the blank was 1 μM. The vehicle consisted of deionized water containing 30 μL of 1 N acetic acid per 50 mL of water. The test compounds and cytisine were dissolved in vehicle. Assays were initiated by vortexing after addition of the membrane suspension to the tube. The samples were incubated at 0 to 4° C. in an iced shaking water bath. Incubations were terminated by rapid filtration under vacuum through Whatman GF/B™ glass fiber filters using a Brandel™ multi-manifold tissue harvester. Following the initial filtration of the assay mixture, filters were washed two times with ice-cold assay buffer (5 m each). The filters were then placed in counting vials and mixed vigorously with 20 ml of Ready Safe™ (Beckman) before quantification of radioactivity. Samples were counted in a LKB Wallach Rackbeta™ liquid scintillation counter at 40-50% efficiency. All determinations were in triplicate.

### Calculations

Specific binding (C) to the membrane is the difference between total binding in the samples containing vehicle only and membrane (A) and non-specific binding in the samples containing the membrane and cytisine (B), i.e.,

Specific binding=(C)=(A)−(B).

Specific binding in the presence of the test compound (E) is the difference between the total binding in the presence of the test compound (D) and non-specific binding (B), i.e., (E)=(D)−(B).

% Inhibition=(1-((E)/(C)) times 100.

The compounds of the invention that were tested in the above assay exhibited IC₅₀ values of less than 10 μM.

What is claimed is:

1. A method of treating a subject suffering from restless legs syndrome comprising administering a nicotinic acetylcholine receptor agonist to the subject in need thereof in an amount effective to treat the syndrome.

2. The method according to claim 1 wherein the nicotinic acetylcholine receptor agonist is a compound of formula I: 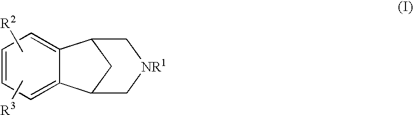
wherein
R¹ is hydrogen, (C₁-C₆)alkyl, unconjugated (C₃-C₆)alkenyl, benzyl, XC(═O)R¹³ or —CH₂CH₂—O—(C₁-C₄)alkyl;
R² and R³ are selected, independently, from hydrogen, (C₂-C₆)alkenyl, (C₂-C₆)alkynyl, hydroxy, nitro, amino, halo, cyano, —SO_{q}(C₁-C₆)alkyl wherein q is zero, one or two, (C₁-C₆)alkylamino—, [(C₁-C₆)alkyl]₂amino—, —CO₂R⁴, —CONR⁵R⁶, —SO₂NR⁷R⁸, —C(═O)R¹³, —XC(═O)R¹³, aryl-(C₀-C₃)alkyl— or aryl-(C₀-C₃)alkyl—O—, wherein said aryl is selected from phenyl and naphthyl, heteroaryl-(C₀-C₃)alkyl— or heteroaryl-(C₀-C₃)alkyl—O—, wherein said heteroaryl is selected from five to seven membered aromatic rings containing from one to four heteroatoms selected from oxygen, nitrogen and sulfur; X²(C₀-C₆)alkyl— and X²(C₁-C₆)alkoxy-(C₀-C₆)alkyl—, wherein X² is absent or X² is (C₁-C₆)alkylamino— or [(C₁-C₆)alkyl]₂amino—, and wherein the (C₀-C₆)alkyl— or (C₁-C₆)alkoxy-(C₀-C₆)alkyl— moieties of said X²(C₀-C₆)alkyl— or X²(C₁-C₆)alkoxy-(C₀-C₆)alkyl— contains at least one carbon atom, and wherein from one to three of the carbon atoms of said (C₀-C₆)alkyl— or (C₁-C₆)alkoxy-(C₀-C₆)alkyl— moieties may optionally be replaced by an oxygen, nitrogen or sulfur atom, with the proviso that any two such heteroatoms must be separated by at least two carbon atoms, and wherein any of the alkyl moieties of said (C₀-C₆)alkyl— or (C₁-C₆)alkoxy-(C₀-C₆)alkyl— groups may be optionally substituted with from two to seven fluorine atoms, and wherein one of the carbon atoms of each of the alkyl moieties of said aryl-(C₀-C₃)alkyl— and said heteroaryl-(C₀-C₃)alkyl— may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆)alkyl optionally substituted with from one to seven fluorine atoms, (C₁-C₆)alkoxy optionally substituted with from two to seven fluorine atoms, halo (e.g., chloro, fluoro, bromo or iodo), (C₂-C₆)alkenyl, (C₂-C₆)alkynyl, hydroxy, nitro, cyano, amino, (C₁-C₆)alkylamino—, [(C₁-C₆)alkyl]₂amino—, —CO₂R⁴, —CONR⁵R⁶, —SO₂NR⁷R⁸, —C(═O)R¹³ and —XC(═O)R¹³;
or R² and R³, together with the carbons to which they are attached, form a four to seven membered monocyclic, or a ten to fourteen membered bicyclic, carbocyclic ring that can be saturated or unsaturated, wherein from one to three of the non-fused carbon atoms of said monocyclic rings, and from one to five of the carbon atoms of said bicyclic rings that are not part of the benzo ring shown in formula I, may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆)alkyl— or (C₁-C₆)alkoxy-(C₀-C₆)alkyl—, wherein the total number of carbon atoms does not exceed six and wherein any of the alkyl moieties may optionally be substituted with from one to seven fluorine atoms; nitro, oxo, cyano, halo, (C₂-C₆)alkenyl, (C₂-C₆)alkynyl, hydroxy, amino, (C₁-C₆)alkylamino—, [(C₁-C₆)alkyl]₂amino—, —CO₂R⁴, —CONR⁵R⁶, —SO₂NR⁷R⁸, —C(═O)R¹³, and —XC(═O)R¹³;
each R⁴, R⁵, R⁶, R⁷, R⁸ and R¹³ is selected, independently, from hydrogen and (C₁-C₆) alkyl, or R⁵ and R⁶, or R⁷ and R⁸ together with the nitrogen to which they are attached, form a pyrrolidine, piperidine, morpholine, azetidine, piperazine, —N—(C₁-C₆)alkylpiperazine or thiomorpholine ring, or a thiomorpholine ring wherein the ring sulfur is replaced with a sulfoxide or sulfone; and
each X is, independently, (C₁-C₆)alkylene;
with the proviso that: (a) at least one of R¹, R² and R³ must be the other than hydrogen, and (b) when R² and R³ are hydrogen, R¹ cannot be hydrogen, (C₁-C₆)alkyl, or unconjugated (C₃-C₆)alkenyl, and pharmaceutically acceptable salts of such compounds.

3. The method according to claim 1 wherein the nicotinic acetylcholine receptor agonist is a compound of formula II 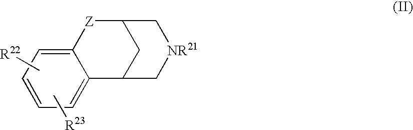
wherein Z is CH₂, C(═O) or CF₂;
R²¹ is hydrogen, (C₁-C₆)alkyl, unconjugated (C₃-C₆)alkenyl, benzyl, XC(═O)R¹³ or —CH₂CH₂—O—(C₁-C₄)alkyl;
R²² and R²³ are selected independently, from hydrogen, (C₂-C₆) alkenyl, (C₂-C₆) alkynyl, hydroxy, nitro, amino, halo, cyano, —SO_{q}(C₁-C₆)alkyl wherein q is zero, one or two, (C₁-C₆)alkylamino, [(C₁-C₆)alkyl]₂amino, CO₂R⁴, CONR⁵R⁶, SO₂NR⁷R⁸, C(═O)R¹³, XC(═O)R¹³, aryl-(C₀-C₃) alkyl or aryl-(C₀-C₃)alkyl-O— wherein said aryl is selected from phenyl and naphthyl, heteroaryl-(C₀-C₃)alkyl or heteroaryl-(C₀-C₃)alkyl-O—, wherein said heteroaryl is selected from five to seven membered aromatic rings containing from one to four heteroatoms selected from oxygen, nitrogen and sulfur, and X²(C₀-C₆)alkoxy-(CO-C₆)alkyl, wherein X² is absent or X² is (C₁-C₆)alkylamino or [(C₁-C₆)alkyl]₂amino, and wherein the (C₀-C₆)alkoxy-(CO-C₆)alkyl moiety of said X²(C₀-C₆)alkoxy-(C₀-C₆)alkyl contains at least one carbon atom, and wherein from one to three of the carbon atoms of said (C₀-C₆)alkoxy-(C₀-C₆)alkyl moiety may optionally be replaced by an oxygen, nitrogen or sulfur atom, with the proviso that any two such heteroatoms must be separated by at least two carbon atoms, and wherein any of the alkyl moieties of said (C₀-C₆)alkoxy-(C₀-C₆)alkyl may be optionally substituted with from two to seven fluorine atoms, and wherein one of the carbon atoms of each of the alkyl moieties of said aryl-(C₀-C₃)alkyl and said heteroaryl-(C₀-C₃)alkyl may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆) alkyl optionally substituted with from one to seven fluorine atoms, (C₁-C₆) alkoxy optionally substituted with from two to seven fluorine atoms, halo (e.g., chloro, fluoro, bromo or iodo), hydroxy, nitro, cyano, amino, (C₁-C₆) alkylamino and [(C₁-C₆) alkyl]₂ amino;
or R²² and R²³, together with the carbons to which they are attached, form a four to seven membered monocyclic, or a ten to fourteen membered bicyclic, carbocyclic ring that can be saturated or unsaturated, wherein from one to three of the nonfused carbon atoms of said monocyclic rings, and from one to five of the carbon atoms of said bicyclic rings that are not part of the benzo ring shown in formula I, may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆) alkoxy-(C₀-C₆)alkyl—, wherein the total number of carbon atoms does not exceed six and wherein any of the alkyl moieties may optionally be substituted with from one to seven fluorine atoms; nitro, oxo, cyano, halo, hydroxy, amino, (C₁-C₆)alkylamino, [(C₁-C₆)alkyl]₂amino, phenyl and monocyclic heteroaryl wherein said heteroaryl is defined as in the definition of R²² and R²³ above;
each R⁴, R⁵, R⁶, R⁷, R⁸, and R¹³ is selected, independently, from hydrogen and (C₁-C₆) alkyl, or R⁵ and R^{6,} or R⁷ and R⁸ together with the nitrogen to which they are attached, form a pyrrolidine, piperidine, morpholine, azetidine, piperazine, —N—(C₁-C₆)alkylpiperazine or thiomorpholine ring, or a thiomorpholine ring wherein the ring sulfur is replaced with a sulfoxide or sulfone; and
each X is, independently, (C₁-C₆)alkylene;
with the proviso that: (a) at least one of R²¹, R²² and R²³ must be the other than hydrogen, (b) when R²² and R²³ are hydrogen, R²¹ cannot be methyl or hydrogen; and (c) no fluorine atom in any of the fluoro substituted alkyl or alkoxy moieties of R²² and R²³ can be attached to a carbon that is attached to a heteroatom;
and the pharmaceutically acceptable salts of such compounds.

4. The method according to claim 2 or 3 wherein the heteroaryl groups within the definition of R² and R³ in formula I or R²² and R²³ in formula II are the following: thienyl, oxazoyl, isoxazolyl, pyridyl, pyrimidyl, thiazolyl, tetrazolyl, isothiazolyl, triazolyl, imidazolyl, tetrazolyl, pyrrolyl and the following groups: 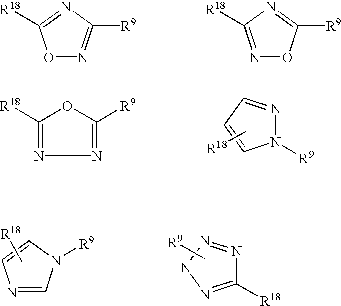
wherein one of R⁹ and R¹⁸ is hydrogen or (C₁-C₆)alkyl, and the other is a bond to the benzo ring of formula I of formula II.

5. The method according to claim 2 or 3 wherein R² and R³ in formula I or R²² and R²³ in formula II, together with the benzo ring of either of formula I or formula II, form a bicyclic ring system selected from the following: 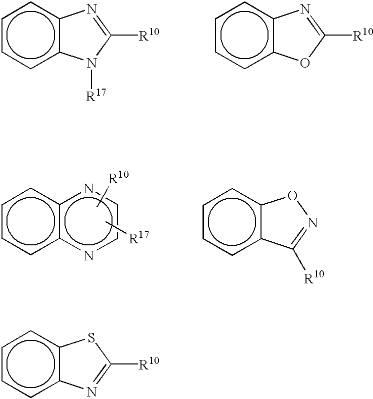
wherein R¹⁰ and R¹⁷ are selected, independently, from hydrogen, (C₁-C₆)alkyl; and (C₁-C₆)alkoxy-(C₀-C₆)alkyl— wherein the total number of carbon atoms does not exceed six and wherein any of the alkyl moieties may optionally be substituted with from one to seven fluorine atoms; nitro, cyano, halo, amino, (C₁-C₆)alkylamino—, [(C₁-C₆) alkyl]₂amino—, —CO₂R⁴, —CONR⁵R⁶, —SO₂NR⁷R⁸, —C(═O)R¹³, —XC(═O)R¹³, phenyl and monocyclic heteroaryl.

6. The method according to claim 2 or 3 wherein R² and R³ in formula I or R²² and R²³ in formula II, together with the benzo ring of formula I or formula II, form a bicyclic or tricyclic ring system selected from the following: 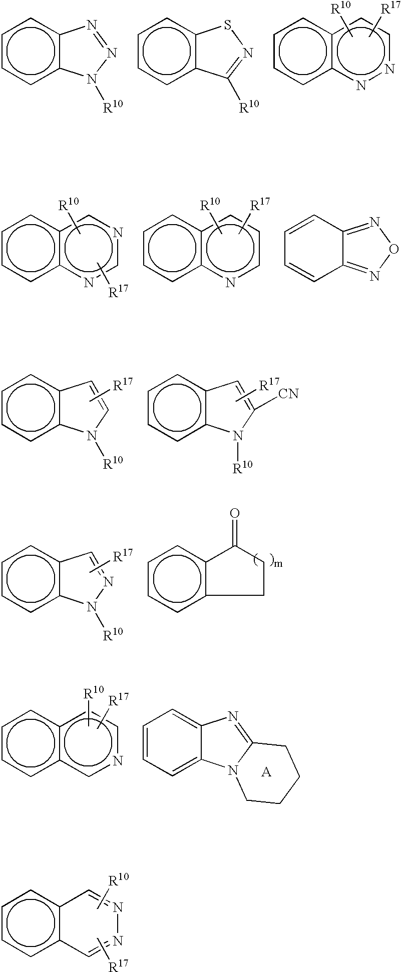
wherein R¹⁰ and R¹⁷ are defined as above, and m is zero, one or two, and wherein one of the carbon atoms of ring A can optionally be replaced with oxygen or N(C₁-C₆)alkyl.

7. The method according to claim 2 or 3 wherein R² and R³ in formula I or R²² or R²³ in formula II do not, together with the benzo ring of formula I or formula II, form a bicyclic or tricyclic ring system.

8. The method according to claim 2 or 3 wherein one of R² and R³ in formula I or R²² or R²³ in formula II is CF₃, fluoro, cyano, (C₂-C₆)alkynyl or C₂F₅.

9. The method according to claim 2 wherein the nicotinic acetylcholine receptor agonist is selected from:
10-aza-tricyclo[6.3.1.0^{2,7}]dodeca-2(7),3,5-triene;
4-fluoro-10-aza-tricyclo[6.3.1.0^{2,7}]dodeca-2(7),3,5-triene;
4-methyl-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
3-trifluoromethyl-10-aza-tricyclo[6.3.1.0^{2,7}]dodeca-2(7),3,5-triene;
3-fluoro-10-aza-tricyclo[6.3.1.0^{2,7}]dodeca-2(7),3,5-triene;
4-nitro-10-azatricyclo[6.3.1.0^{2,7}]dodeca-2(7),3,5-triene;
4-amino-10-azatricyclo[6.3.1.0^{2,7}]dodeca-2(7),3,5-triene;
N¹-[10-azatricyclo[6.3.1.0^{2,7}]dodeca-2(7),3,5-trien-4-yl]-acetamide;
6-methyl-5-thia-7,13-diazatetracyclo[9.3.1.0^{2,10}.0^{4,8}]pentadeca-2(10),3,6,8-tetraene;
6-methyl-7-propyl-5,7,13-triazatetracyclo[9.3.1.0^{2,10}.0^{4,8}]-pentadeca-2(10),3,5,8-tetraene;
5,7,13-triazatetracyclo[9.3.10^{2,10}.0^{4,8}]-pentadeca-2(10),3,5,8-tetraene;
7-methyl-5,7,13-triazatetracyclo[9.3.10^{2,10}.0^{4,8}]-pentadeca-2(10),3,5,8-tetraene;
6-methyl-5,7,13-triazatetracyclo[9.3.1.0^{2,10}.0^{4,8}]-pentadeca-2(10),3,5,8-tetraene;
6,7-dimethyl-5,7,13-triazatetracyclo[9.3.1.0^{2,10}.0^{4,8}]-pentadeca-2(10),3,5,8-tetraene;
7-propyl-5,7,13-triazatetracyclo[9.3.1.0^{2,10}.0^{4,8}]-pentadeca-2(10),3,5,8-tetraene;
7-butyl-5,7,13-triazatetracyclo[9.3.1.0^{2,10}.0^{4,8}]-pentadeca-2(10),3,5,8-tetraene;
7-isobutyl-5,7,13-triazatetracyclo[9.3.1.0^{2,10}.0^{4,8}]-pentadeca-2(10),3,5,8-tetraene;
6-methyl-7-isobutyl-5,7,13-triazatetracyclo[9.3.1.0^{2,10}.0^{4,8}]-pentadeca-2(10),3,5,8-tetraene;
7-phenyl-5,7,13-triazatetracyclo[9.3.1.0^{2,10}.0^{4,8}]-pentadeca-2( 10),3,5,8-tetraene;
6-methyl-7-phenyl-5,7,13-triazatetracyclo[9.3.1.0^{2,10}.0^{4,8}]-pentadeca-2(10),3,5,8-tetraene;
6-methyl-7-neopentyl-5,7,13-triazatetracyclo[9.3.1.0^{2,10}.0^{4,8}]-pentadeca-2(10),3,5,8-tetraene;
6,7-dimethyl-5,8,14-triazatetracyclo[10.3.10^{2,11}.0^{4,9}]-hexadeca-2(11),3,5,7,9-pentaene;
5,8,14-triazatetracyclo[10.3.1.0^{2,11}.0^{4,9}]-hexadeca-2(11),3,5,7,9-pentaene;
14-methyl-5,8,14-triazatetracyclo[10.3.1.0^{2,11}.0^{4,9}]-hexadeca-2(11),3,5,7,9-pentaene;
5-oxa-7,13-diazatetracyclo[9.3.1.0^{2,10}.0^{4,8}]-pentadeca-2(10),3,6,8-tetraene;
6-methyl-5-oxa-7,13-diazatetracyclo[9.3.1.0^{2,10}.0^{4,8}]-pentadeca-2(10),3,6,8-tetraene;
2-fluoro-N-(4-hydroxy-10-aza-tricyclo[6.3.1.0^{2,7}]-dodeca-2(7),3,5-trien-5-yl)-benzamide;
4-chloro-10-azatricyclo[6.3.1.0^{2,7}]dodeca-2(7),3,5-triene;
10-azatricyclo[6.3.1.0^{2,7} ]dodeca-2(7),3,5-trien-4-yl cyanide;
3-(10-azatricyclo[6.3.1.0^{2,7}]dodeca-2(7),3,5-trien-4-yl)-5-methyl-1,2,4-oxadiazole;
1-(10-azatricyclo[6.3.1.0^{2,7}]dodeca-2(7),3,5-trien-4-yl)-1-ethanone;
10-azatricyclo[6.3.1.0^{2,7}]dodeca-2(7),3,5-trien-4-ol;
7-methyl-5-oxa-6,13-diazatetracyclo[9.3.1.0^{2,10}.0^{4,8}]pentadeca-2,4(8),6,9-tetraene;
4-(2-methyl-2H-pyrazol-3-yl)-10-aza-tricyclo[6.3.1.0^{2,7}]dodeca-2(7),3,5-triene;
4-(1-methyl-1H-pyrazol-3-yl)-10-aza-tricyclo[6.3.1.0^{2,7}]dodeca-2(7),3,5-triene;
4,5-dichloro-10-azatricyclo[6.3.1.0^{2,7}]dodeca-2(7),3,5-triene;
N⁴, N⁴-dimethyl-10-azatricyclo[6.3.1.0^{2,7}]-dodeca-2(7),3,5-triene-4-sulfonamide;
4-(1-pyrrolidinylsulfonyl)-10-azatricyclo[6.3.1.0^{2,7}]-dodeca-2(7),3,5-triene;
5,13-diazatetracyclo[9.3.1.0^{2,10}.0^{4,8}]-pentadeca-2,4(8),9-trien-6-one;
6-oxo-5-oxa-7,13-diazatetracyclo[9.3.1.0^{2,10}.0^{4,8}]-pentadeca-2(10),3,6,8-tetraene;
3-phenyl-10-aza-tricyclo[6.3.1.0^{2,7}]dodeca-2(7),3,5-triene;
3-hydroxy-10-aza-tricyclo[6.3.1.0^{2,7}]dodeca-2(7),3,5-triene;
4,5-difluoro-10-aza-tricyclo[6.3.1.0^{2,7}]dodeca-2(7),3,5-triene;
6-ethyl-5-oxa-7,13-diazatetracyclo[9.3.1.0^{2,10}.0^{4,8}]-pentadeca-2(10),3,6,8-tetraene;
6-isopropyl-5-oxa-7,13-diazatetracyclo[9.3.1.0^{2,10}.0^{4,8}]-pentadeca-2(10),3,6,8-tetraene;
6-benzyl-5-oxa-7,13-diazatetracyclo[9.3.1.0^{2,10}.0^{4,8}]-pentadeca-2(10),3,6,8-tetraene;
5,14-diazatetracyclo[10.3.1.0^{2,11}.0^{4,9}]hexadeca-2(11),3,5,7,9-pentaene;
6-methyl-5,14-diazatetracyclo[10.3.1.0^{2,11}.0^{4,9}]hexadeca-2(11),3,5,7,9-pentaene;
7-methyl-5,14-diazatetracyclo[10.3.1.0^{2,11}.0^{4,9}]hexadeca-2(11),3,5,7,9-pentaene;
7-ethyl-5,14-diazatetracyclo[10.3.1.0^{2,11}.0^{4,9}]hexadeca-2(11),3,5,7,9-pentaene;
8-methyl-5,14-diazatetracyclo[10.3.1.0^{2,11}.0^{4,9}]hexadeca-2(11),3,5,7,9-pentaene;
5,14-diazatetracyclo[10.3.1.0^{2,11}.0^{4,9}]hexadeca-2(11),3,7,9-tetraen-6-one;
6-chloro-5,14-diazatetracyclo[10.3.1.0^{2,11}.0^{4,9}]hexadeca-2(11),3,5,7,9-pentaene;
6-methoxy-5,14-diazatetracyclo[10.3.1.0^{2,11}.0^{4,9}]hexadeca-2(11),3,5,7,9-pentaene;
6-chloro-10-fluoro-5,14-diazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,7,9-tetraen-6-one;
and pharmaceutically acceptable salts and optical isomers thereof.

10. The method according to claim 3 wherein the nicotinic acetylcholine receptor agonist is selected from:
5,6-difluoro-11-azatricyclo[7.3.1.0^{2,7}]trideca-2 4,6-triene;
11-benzyl-6-methoxy-11-aza-tricyclo[7.3.1.0^{2,7}]trideca-2(7),3,5-triene;
6-methoxy-11-aza-tricyclo[7.3.1.0^{2,7}]trideca-2(7),3,5-triene;
11-aza-tricyclo[7.3.1.0^{2,7}]trideca-2(7),3,5-trien-6-ol;
6-fluoro-11-aza-tricyclo[7.3.1.0^{2,7}]trideca-2(7),3,5-triene;
11-benzyl-5-methoxy-11-aza-tricyclo[7.3.1.0^{2,7}]trideca-2(7),3,5-triene;
11-benzyl-11-aza-tricyclo[7.3.1.0^{2,7}]trideca-2(7),3,5-trien-5-ol;
5-methoxy-11-aza-tricyclo[7.3.1.0^{2,7}]trideca-2(7),3,5-triene;
11-aza-tricyclo[7.3.1.0^{2,7}]trideca-2(7),3,5-trien-5-ol;
11-benzyl-5-difluoromethoxy-11-aza-tricyclo[7.3.1.0^{2,7}]trideca-2(7),3,5-triene;
5-difluoromethoxy-11-aza-tricyclo[7.3.1.0^{2,7}]trideca-2(7),3, 5-triene;
11-benzyl-5-ethoxy-11-aza-tricyclo[7.3.1.0^{2,7}]trideca-2(7),3,5-triene;
5-ethoxy-11-aza-tricyclo[7.3.1.0^{2,7}]trideca-2(7),3,5-triene;
5-isopropoxy-11-aza-tricyclo[7.3.1.0^{2,7}]trideca-2(7),3,5-triene;
11-benzyl-4-methoxy-11-aza-tricyclo[7.3.1.0^{2,7}]trideca-2(7),3,5-triene;
4-methoxy-11-aza-tricyclo[7.3.1.0^{2,7}]trideca-2(7),3,5-triene;
11-aza-tricyclo[7.3.1.0^{2,7}]trideca-2(7),3,5-trien-4-ol;
11-benzyl-11-aza-tricyclo[7.3.1.0^{2,7}]trideca-2(7),3,5-triene;
4-nitro-11-aza-tricyclo[7.3.1.0^{2,7}]trideca-2(7),3,5-triene;
5-nitro-11-aza-tricyclo[7.3.1.0^{2,7}]trideca-2(7),3,5-triene;
3-nitro-11-aza-tricyclo[7.3.1.0^{2,7}]trideca-2(7),3,5-triene;
11-benzyl-5-fluoro-11-aza-tricyclo[7.3.1.0^{2,7}]trideca-2(7),3,5-triene;
5-fluoro-11-aza-tricyclo[7.3.1.0^{2,7}]trideca-2(7),3,5-triene;
5,7-dioxa-14-azatetracyclo[10.3.1.0^{2,10}.0^{4,8}]hexadeca-2(10),3,8-triene;
11-benzyl-6-bromo-5-methoxy-11-aza-tricyclo[7.3.1.0^{2,7}]trideca-2(7),3,5-triene;
11-benzyl-6-hydroxy-5-methoxy-11-aza-tricyclo[7.3.1.0^{2,7}]trideca-2(7),3,5 -triene;
6-hydroxy-5-methoxy-11-aza-tricyclo[7.3.1.0^{2,7}]trideca-2(7),3,5 -triene;
trifluoromethanesulfonic acid-11-benzyl-11-aza-tricyclo[7.3.1.0^{2,7}]trideca-2(7),3,5-trien-5-yl ester;
5-(4-trifluoromethyl-phenyl)-11-aza-tricyclo[7.3.1.0^{2,7}]trideca-2(7),3,5-triene;
5-(4-methoxy-phenyl)-11-aza-tricyclo[7.3.1.0^{2,7}]trideca-2(7),3,5-triene;
11-aza-tricyclo[7.3.1.0^{2,7}]trideca-2(7),3,5-triene-5-carboxylic acid methyl ester;
2-(11-aza-tricyclo[7.3.1.0^{2,7}]trideca-2(7),3,5-trien-5-yl)-propan-2-ol;
5-pyridin-3-yl-11-aza-tricyclo[7.3.1.0^{2,7}]trideca-2(7),3,5-triene;
and pharmaceutically acceptable salts and optical isomers thereof.

11. The method according to claim 1 wherein the nicotinic acetylcholine receptor agonist is a compound of formula III 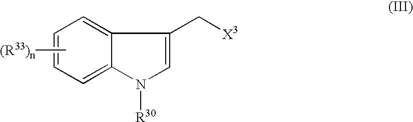
wherein X³ is:
a) —CH₂NR³¹R³²,
b) 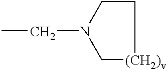
or
c) 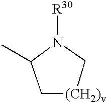
wherein R³⁰ R³¹, and R³² are independently selected from hydrogen and C₁-C₆ alkyl;
R³³ is selected from hydrogen, halogen and C₁-C₆ alkyl;
v is an integer from 0 to 4; and
n is an integer from 0 to 2; and pharmaceutically acceptable salts thereof.

12. The method according to claim 11 wherein the nicotinic acetylcholine receptor agonist is selected from the group consisting of:
[2-(6-chloro-1H-pyrrolo[2,3-b]pyridin-3-yl)ethyl]-dimethylamine;
[2-(6-chloro-1H-pyrrolo[2,3-b]pyridin-3-yl)ethyl]-methylamine;
3-pyrrolidin-2-ylmethyl-1H-pyrrolo[2,3-b]pyridine;
3-(1-methyl-pyrrolidin-2-ylmethyl)-1-H-pyrrolo[2,3-b]pyridine;
dimethyl-[2-(1H-pyrrolo[2,3-b]pyridin-3-yl)-ethyl]-amine;
methyl-[2-(1H-pyrrolo[2,3-b]pyridin-3-yl)-ethyl]-amine;
2-(1H-pyrrolo[2,3-b]pyridin-3-yl-ethylamine; and
3-(2-piperidin-1-yl-ethyl-1H-pyrrolo[2,3-b]pyridine.
and pharmaceutically acceptable salts and optical isomers thereof.

13. The method according to claim 1 wherein the nicotinic acetylcholine receptor agonist is a compound of formula IV: 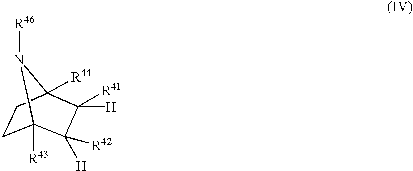
wherein R⁴¹, R⁴², R⁴³ and R⁴⁴ are selected, independently from hydrogen, —CO₂R⁴⁵, aryl and heteroaryl, wherein said aryl is selected from phenyl and naphthyl and said heteroaryl is selected from pyrazinyl, benzofuranyl, quinolyl, isoquinolyl, benzothienyl, isobenzofuryl, pyrazolyl, indolyl, isoindolyl, benzimidazolyl, purinyl, carbazolyl, 1,2,5-thiadiazolyl, quinazolinyl, pyridazinyl, pyrazinyl, cinnolinyl, phthalazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidiny, and pyrazolopyrimidinyl oxazolyl, isoxazoyl, thiazolyl, isothiazolyl, furanyl, pyrazolyl, pyrrolyl, tetrazolyl, triazolyl, thienyl, imidazolyl, pyridinyl, and pyrimidinyl, and wherein said phenyl and said heteroaryl may optionally be substituted with from one to three substituents, and are preferably substituted with one or two substituents, independently selected form (C₁-C₆)alkyl optionally substituted with from one to seven (preferably with from zero to four) fluorine atoms, halo (i.e., chloro, fluoro, bromo or iodo), phenyl, benzyl, hydroxy, acetyl, amino, cyano, nitro, (C₁-C₆)alkoxy optionally substituted with from one to seven (preferably with from zero to four) fluorine atoms, (C₁-C₆)alkylamino and [(C₁-C₆)alkyl]₂amino;
R⁴⁵ is (C₁-C₆) alkyl, aryl, heteroaryl, (C₁-C₄)alkylene-aryl and (C₁-C₄)alkylene-heteroaryl, wherein said aryl and heteroaryl are defined as above, and wherein said (C₁-C₆)alkyl may optionally be substituted with from one to three substituents independently selected from halo, (C₁-C₆)alkyl, (C₁-C₆ )alkoxy, (C₁-C₄)alkoxy-(C₁-C₄)alkyl, amino, (C₁-C₆)alkylamino, and [(C₁-C₆)alkyl]₂amino; and
R⁴⁶ is hydrogen or (C₁-C₆)alkyl;
with the proviso that: (a) at least one of R⁴¹, R^{42,} R^{43,} and R⁴⁴ must be aryl or heteroaryl; (b) when neither R⁴¹ nor R⁴² is hydrogen, R⁴¹ and R⁴² are in the “exo” configuration; (c) R⁴¹ and R⁴² can not both be —CO₂R⁴⁵; (d) if either R⁴³ or R⁴⁴ is —CO₂R⁴⁵ and R⁴⁵ is an alkyl or alkoxyalkyl group, then one of R⁴¹ and R⁴² must be aryl or heteroaryl; and (e) if either R⁴¹ or R⁴² is —CO₂R⁴⁵ and R⁴⁵ is an alkyl or alkoxyalkyl group, then one of R⁴³ and R⁴⁴ must be aryl or heteroaryl;
and the pharmaceutically acceptable salts of such compounds.

14. The method according to claim 13 wherein one of R⁴¹ and R⁴² is optionally substituted phenyl and the other is hydrogen, and wherein R⁴³ and R⁴⁴ are hydrogen.

15. The method according to claim 13 wherein one of R⁴¹ and R⁴² is phenyl substituted with fluoro or nitro and the other is hydrogen, and wherein R⁴³ and R⁴⁴ are hydrogen.

16. The method according to claim 13 wherein R⁴³ and R⁴⁴ are hydrogen and one R⁴¹ and R⁴² is hydrogen and the other is: (a) 3-fluorophenyl; (b) 4-nitrophenyl; or 3-fluoro-4-nitrophenyl.

17. The method according to claim 13 wherein the nicotinic acetylcholine receptor agonist is selected from:
2β-(3,4-difluorophenyl)-7-aza-bicyclo[2.2.1]heptane;
2β-(3,5-dichlorobenzene)-7-aza-bicyclo[2.2.1]heptane;
2β-(4-nitrophenyl)-7-aza-bicyclo[2.2.1]heptane;
2β-(3-thiophene)-7-aza-bicyclo[2.2.1]heptane;
2β-(3-fluoro-4-chlorophenyl)-7-aza-bicyclo[2.2.1]heptane;
2β-(3-flourophenyl)-7-aza-bicyclo[2.2.1]heptane;
2β-(3-hydroxyphenyl)-7-aza-bicyclo[2.2.1]heptane;
2β-(3-acetophenone)-7-aza-bicyclo[2.2.1]heptane;
2β-(4-trifluoromethylphenyl)-7-aza-bicyclo[2.2.1]heptane;
2β-(3-fluoro-4-methylphenyl)-7-aza-bicyclo[2.2.1]heptane;
2β-(3-chlorophenyl)-7-aza-bicyclo[2.2.1]heptane;
2β-(n-benzyl-5-pyridonyl)-7-aza-bicyclo[2.2.1]heptane;
2β-(n-methyl-5-pyridonyl)-7-aza-bicyclo[2.2.1]heptane;
2β-(3-fluoro-5-nitrophenyl)-7-aza-bicyclo[2.2.1]heptane;
2β-(4-aminophenyl)-7-aza-bicyclo[2.2.1]heptane;
2β-(3-fluoro-4-trifluoromethyl-phenyl)-7-aza-bicyclo[2.2.1]heptane;
2β-(4-chlorophenyl)-7-aza-bicyclo[2.2.1]heptane;
2β-(3,4-methylenedioxyphenyl)-7-aza-bicyclo[2.2.1]heptane;
2β-(2-chloro-6-methyl-5-pyridinyl)-7-aza-bicyclo[2.2.1]heptane;
2β-(4-cyanophenyl)-7-aza-bicyclo[2.2.1]heptane;
2β-(3-fluoro-4-nitro-phenyl)-7-aza-bicyclo[2.2.1]heptane;
2β-(4-amido-phenyl)-7-aza-bicyclo[2.2.1]heptane;
2β-(3-fluoro-4-amino-phenyl)-7-aza-bicyclo[2.2.1]heptane;
2β-(4-sulfonamido-phenyl)-7-aza-bicyclo[2.2.1]heptane;
2β-(3-methyl-5-isoxzazole)-7-aza-bicyclo[2.2.1]heptane;
2β-(3-methyl-5-isoxzazole)-7-aza-bicyclo[2.2.1]heptane, N-methyl;
2β-(3-methyl-5-isoxzazole)-7-aza-bicyclo[2.2.1]heptane, N-acetyl;
2b-(3,4-difluorophenyl)-7-azabicyclo[2.2.1]heptane;
4-(7-aza-bicyclo[2.2.1]hept-2-yl)-benzamidine;
2-(4-methanesulfonyl-phenyl)-7-aza-bicyclo[2.2.1]heptane;
4-(7-aza-bicyclo[2.2.1]hept-2-yl)-phenol;
2-(4-methylsulfanyl-phenyl)-7-aza-bicyclo[2.2.1]heptane;
4-(7-aza-bicyclo[2.2.1]hept-2-yl)-benzoic acid methyl ester;
4-(7-aza-bicyclo[2.2.1]hept-2-yl)-benzoic acid;
2-(3-fluoro-4-tetrazol-1-yl-phenyl)-7-aza-bicyclo[2.2.1]heptane;
2-(4-nitro-3-trifluoromethyl-phenyl)-7-aza-bicyclo[2.2.1]heptane;
2-[3-fluoro-4-(5-trifluoromethyl-tetrazol-1-yl)-phenyl]-7-aza-bicyclo[2.2.1]heptane;
2-(3-chloro-4-nitro-phenyl)-7-aza-bicyclo[2.2.1]heptane;
2-(4-tetrazol-1-yl-phenyl)-7-aza-bicyclo[2.2.1]heptane;
2-(6-methoxy-pyridin-2-yl)-7-aza-bicyclo[2.2.1]heptane;
2-(4-methanesulfinyl-phenyl)-7-aza-bicyclo[2.2.1]heptane;
2-(4-bromo-3-fluoro-phenyl)-7-aza-bicyclo[2.2.1]heptane;
2-(4-cyano-3-fluoro-phenyl)-7-aza-bicyclo[2.2.1]heptane;
2-(3,4,5-trifluoro-phenyl)-7-aza-bicyclo[2.2.1]heptane;
2-(3,4,5-trimethoxy-phenyl)-7-aza-bicyclo[2.2.1]heptane;
2-(5-nitro-furan-2-yl)-7-aza-bicyclo[2.2.1]heptane;
5-(7-aza-bicyclo[2.2.1]hept-2-yl)-3-methyl-benzo[d]isoxazole;
6-(7-aza-bicyclo[2.2.1]hept-2-yl)-3-methyl-benzo[d]isoxazole;
6-(7-aza-bicyclo[2.2.1]hept-2-yl)-1,4-dihydro-quinoxaline-2,3-dione;
6-(7-aza-bicyclo[2.2.1]hept-2-yl)-quinoxaline; and
1-[4-(7-aza-bicyclo[2.2.1]hept-2-yl)-2-fluoro-phenyl]-ethanone
and pharmaceutically acceptable salts and optical isomers thereof.

18. The method according to claim 1 wherein the nicotinic acetylcholine receptor agonist is a compound of formula V: 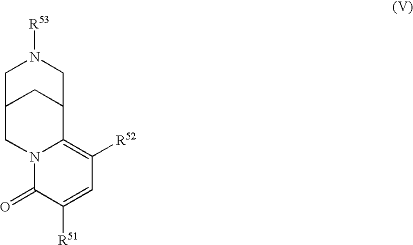
its enantiomers, diastereomers and stereoisomers, and their pharmaceutically acceptable salts and prodrugs,
wherein R⁵¹ and R⁵² are each independently selected from
a) H; halo; CF₃; hydroxy; (C₁-C₆)alkoxy; CH₂OH; —C(O)R^{54,} wherein R⁵⁴ is H, (C₁-C₆)alkyl, (C₆-C₁₀)aryl or benzyl (including substituted alkyl, aryl or benzyl); C≡N; C≡CR⁵⁵, wherein R⁵⁵ is H, (C₁-C₆)alkyl, (C₆-C₁₀)aryl (including substituted alkyl or aryl); —S(O)ₙR⁵⁵, wherein R⁵⁵ is H, (C₁-C₆)alkyl, or (C₆-C₁₀)aryl (including substituted alkyl or aryl) and n is 0, 1, or 2; (C₁-C₆)alkyl; (C₁-C₆)alkenyl; H₂N; di-((C₁-C₆)alkyl)amino; mono(C₁-C₆)alkyl-amino; (C₆-C₁₀)aryl-amino; (C₃-C₈)cycloalkyl-amino; heteroaryl-amino; cycloheteroalkyl-amino; and CON(R⁵⁵)₂ wherein each R⁵⁵ is selected from hydrogen, (C₁-C₆)alkyl and (C₆-C₁₀)aryl; and
b) CO₂R⁵⁶ wherein R⁵⁶ is selected from H, (C₁-C₆)alkyl, phenyl and benzyl; and
c) optionally benzene-fused (C₆-C₁₀)aryl, optionally benzene-fused (C₃-C₈)cycloalkyl, optionally benzene-fused heteroaryl and optionally benzene-fused cycloheteroalkyl, wherein said heteroaryl group contains five to ten atoms comprising one to four heteroatoms, said cycloheteroalkyl contains 4 to 8 atoms comprising one or two heteroatoms selected from N, S and O;
and wherein any of the alkyl, alkenyl, aryl, cycloalkyl, cycloheteroalkyl and heteroaryl groups in a), b) and c) are optionally substituted with one or more substituents selected from halogen, (C₁-C₆)alkyl, (C₆-C₁₀)aryl, hydroxy, hydroxymethyl, CHO and CO₂R⁵⁶ wherein R⁵⁶ is as described above; and
R⁵³ is selected from H, optionally substituted benzyl and methyl;
with the provisos that R⁵¹ and R⁵² are not both hydrogen and when R⁵³ is H, and that R⁵¹ and R⁵² when selected from H, Br and Cl are not be the same.

19. The method according to claim 18 wherein R⁵³ is selected from H, benzyl or methyl and R⁵¹ and R⁵² are each independently selected from H, halo, (C₁-C₆)alkyl, cyano, (C₆-C₁₀)aryl, (C₅-C₉)heteroaryl, (C₁-C₆)alkenyl, (C₂-C₆)alkynyl-R⁵⁵ and —C(O)R⁵⁵ wherein R⁵⁵ is H, (C₁-C₆) alkyl, (C₆-C₁₀)aryl and (C₅-C₉)heteroaryl and amino and mono and di-substituted amino; with the provisos that when R⁵³ is H then R⁵¹ and R⁵² are not both H, Br and Cl and when R⁵³ is benzyl or methyl, then R⁵¹ and R⁵² are not hydrogen.

20. The method according to claim 18 wherein R⁵¹ and R⁵² are each independently selected from H, ethyl, methyl, phenyl, vinyl, fluoro, bromo, chloro, isopropyl, tert-butyl, trifluoromethyl, acetyl, propanoyl, 2,2-dimethylpropanoyl, 2-methylpropanoyl, butanoyl, pentanoyl, cyano, di-[(C₁-C₆)alkyl]amino, (C₁-C₆)monoalkylamino, (C₆-C₁₀)arylamino, (C₃-C₈)cycloalkylamino, heteroarylamino, cycloheteroalkyamino and CON(R⁵⁵)₂ wherein each R⁵⁵ is selected from hydrogen, (C₁-C₆)alkyl and (C₆-C₁₀)aryl; (C₆-C₁₀)aryl and (C₅-C₉)heteroaryl wherein the aryl and heteroaryl groups are optionally substituted with one or more substituents selected from halogen, (C₁-C₆)alkyl, (C₆-C₁₀)aryl, hydroxy, hydroxymethyl, CHO and CO₂R⁵⁶ wherein R⁵⁶ is selected from H, (C₁-C₆)alkyl, phenyl and benzyl.

21. The method according to claim 18 wherein R⁵³ is selected from optionally substituted benzyl or (C₁-C₆)alkyl, wherein the substituents are described above and R⁵¹ and R⁵² are each independently selected from hydrogen, halo, cyano, optionally substituted (C₁-C₆)alkyl, (C₁-C₆)alkenyl, amino, di-[(C₁-C₆)alkyl]amino, (C₁-C₆)monoalkylamino, (C₆-C₁₀)arylamino, (C₃-C₈)cycloalkylamino, heteroarylamino, cycloheteroalkyamino and CON(R⁵⁵)₂ wherein each R⁵⁵ is selected from hydrogen, (C₁-C₆)alkyl and (C₆-C₁₀)aryl; -C(O)R⁵⁵ wherein R⁵⁵ is H, (C₁-C₆)alkyl, or (C₆-C₁₀)aryl; (C₆-C₁₀)aryl or (C₅-C₉)heteroaryl wherein the substituents are described above.

22. The method according to claim 18 wherein R⁵¹ and R⁵² are each independently selected from hydrogen isopropyl, tert-butyl, trifluoromethyl, acetyl, propanoyl, 2,2-dimethylpropanoyl, 2-methylpropanoyl, butanoyl, pentanoyl, cyano, 2,4-difluorophenyl, 2-fluorophenyl, 2- and 3-thienyl, dimethylamino and R⁵³ is selected from hydrogen, benzyl, methyl and R⁵¹ and R⁵² are each independently selected from hydrogen, bromo, chloro, ethyl, methyl, fluoro, vinyl and phenyl.

23. The method according to claim 18 wherein the nicotinic acetylcholine receptor agonist is selected from:
9-bromo-1,2,3,4,5,6-hexahydro-1,5-methano-pyrido[1,2-a][1,5]diazocin-8-one;
11-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
11-chloro-1,2,3,4,5,6-hexahydro-1,5-methano-pyrido[1,2-a][1,5]diazocin-8-one;
9-flouro-1,2,3,4,5,6-hexahydro-1,5-methano-pyrido[1,2-a][1,5]diazocin-8-one;
11-flouro-1,2,3,4,5,6-hexahydro-1,5-methano-pyrido[1,2-a][1,5]diazocin-8-one;
9,11-diflouro-1,2,3,4,5,6-hexahydro-1,5-methano-pyrido[1,2-a][1,5]diazocin-8-one;
9-ethyl-1,2,3,4,5,6-hexahydro-1,5-methano-pyrido[1,2-a][1,5]diazocin-8-one;
11-ethyl-1,2,3,4,5,6-hexahydro-1,5-methano-pyrido[1,2-a][1,5]diazocin-8-one;
9,11-diethyl-1,2,3,4,5,6-hexahydro-1,5-methano-pyrido[1,2-a][1,5]diazocin-8-one;
9-methyl-1,2,3,4,5,6-hexahydro-1,5-methano-pyrido[1,2-a][1,5]diazocin-8-one;
11-methyl-1,2,3,4,5,6-hexahydro-1,5-methano-pyrido[1,2-a][1,5]diazocin-8-one;
9,11-dimethyl-1,2,3,4,5,6-hexahydro-1,5-methano-pyrido[1,2-a][1,5]diazocin-8-one;
9-phenyl-1,2,3,4,5,6-hexahydro-1,5-methano-pyrido[1,2-a][1,5]diazocin-8-one;
11-phenyl-1,2,3,4,5,6-hexahydro-1,5-methano-pyrido[1,2-a][1,5]diazocin-8-one;
9,11-diphenyl-1,2,3,4,5,6-hexahydro-1,5-methano-pyrido[1,2-a][1,5]diazocin-8-one ;
9-vinyl-1,2,3,4,5,6-hexahydro-1,5-methano-pyrido[1,2-a][1,5]diazocin-8-one;
11-vinyl-1,2,3,4,5,6-hexahydro-1,5-methano-pyrido[1,2-a][1,5]diazocin-8-one;
9,11-divinyl-1,2,3,4,5,6-hexahydro-1,5-methano-pyrido[1,2-a][1,5]diazocin-8-one;
9-bromo-3-methyl-1,2,3,4,5,6-hexahydro-1,5-methano-pyrido[1,2-a][1,5]diazocin-8-one;
3-benzyl-9-bromo-1,2,3,4,5,6-hexahydro-1,5-methano-pyrido[1,2-a][1,5]diazocin-8-one; and
3-benzyl-9-chloro-1,2,3,4,5,6-hexahydro-1,5-methano-pyrido[1,2-a][1,5]diazocin-8-one
and pharmaceutically acceptable salts and optical isomers thereof.

24. The method according to claim 1 wherein the nicotinic acetylcholine receptor agonist is a compound of formula VI: 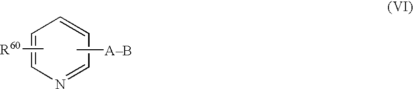
and their pharmaceutically acceptable acid addition salts and prodrugs,
wherein A is —CH(R⁶¹ )— and R⁶¹ is hydrogen or optionally substituted (C₁-C₆)alkyl wherein the substituents comprise one or more groups individually selected from hydroxy, (C₁-C₆)alkoxy, oxo, (C₂-C₆)alkanoyl and NR⁶²R⁶³; and
B is a group of the formula 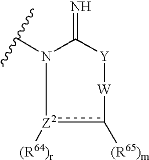
wherein Y—W is CH₂, NH, O, S, CH₂CH₂, CH═CH, N═CH, NH—CH₂, OCH₂ or SCH₂;
the dotted line represents an optional bond;
Z² is C, N, O or S;
m is 1 or 2;
r is 0, 1 or 2 with the proviso that r is 0 when Z² is O or S, r is 1 when Z² is N and r is 2 when Z² is C;
each R⁶⁴ and R⁶⁵ is independently selected from hydrogen, optionally substituted (C₁-C₆)alkyl, optionally substituted (C₁-C₆)alkoxy and optionally substituted (C₂-C₆)alkanoyl, wherein the substituents on the alkyl or alkanoyl groups are selected from hydroxy, (C₁-C₆)alkoxy, oxo, (C₂-C₆)alkanoyl and NR⁶²R⁶³, or R⁶⁴ and R⁶⁵ together with the carbon atoms to which they are attached form an optionally substituted six membered heteroaromatic ring containing at least one heteroatom selected from N, S and O and Z² is C wherein said substituents are selected from optionally substituted (C₁-C₆)alkyl or optionally substituted (C₁-C₆)alkoxy wherein said substituents are selected from (C₁-C₆)alkyl, optionally substituted (C₁-C₆)alkoxy and optionally substituted (C₂-C₆)alkanoyl or R⁶⁴ and one of R⁶⁵ together form a bond with the proviso that R⁶⁴ and R⁶⁵ cannot form a bond when Z² is O or S;
R⁶⁰ is hydrogen or halo; and
R⁶² and R⁶³ are each independently selected from hydrogen and optionally substituted (C₁-C₆)alkyl wherein said substituents are selected from (C₁-C₆)alkyl and halo;
with the provisos that when —B—A is attached to the 3-position of the pyridine ring and R⁶¹ is hydrogen and
a) R⁶⁰ is 6-chloro and
i) Z² is C, the dotted line represents a bond, m and r are both 1, R⁶⁴ and R⁶⁵ are both hydrogen, then W—Y is not selected from CH═CH, S, CH₂, NH, CH═N, OCH₂ or SCH₂;
ii) Z² is nitrogen, the dotted line represents a bond, r is 0 and m is 1 then R⁶⁵ is not CF₃; or
iii) Z² is C, the dotted line represents a bond, m and r are both 2, and each R⁶⁴ and R⁶⁵ is hydrogen, then W—Y is not S; or
b) R⁶⁰ is hydrogen, 6-bromo or 6-fluoro and Z² is carbon, the dotted line represents a bond, m and r are both 1, R⁶⁴ and R⁶⁵ are both hydrogen, then W—Y is not sulfur.

25. The method according to claim 24 wherein the nicotinic acetylcholine receptor agonist is selected from:
3-(6-chloro-pyridin-3-ylmethyl)-3H-[1,3,4]thiadiazol-2-ylideneamine;
5-methyl-3-pyridin-3-ylmethyl-3H-thiazol-2-ylideneamine;
3-(6-chloro-pyridin-3-ylmethyl)-5-methyl-3H-[1,3,4]thiadiozol-2-ylideneamine;
6-chloro-2-(6-chloro-pyridin-3-ylmethyl)-2H-pyridazin-3-ylideneamine;
3-(6-chloro-pyridin-3-ylmethyl)-3H-benzothiazol-2-ylideneamine;
3-pyridin-3-ylmethyl-3H-[1,3,4]thiadiazol-2-ylideneamine;
3-[1-(6-chloro-pyridin-3-yl)-ethyl]-3H-thiazol-2-ylideneamine;
3-[1-(6-chloro-pyridin-3-yl)-ethyl]-3H-[1,3,4]thiadiazol-2-ylideneamine;
3-[1-(6-chloro-pyridin-3-ylmethyl)-thiazolidin-2-ylideneamine;
3-pyridin-3-ylmethyl-thiazolidin-2-ylideneamine;
5,7-dimethyl-1-pyridin-3-ylmethyl-3H-[1,8]naphthyridin-2-ylidene;
6-chloro-2-pyridin-3-ylmethyl-2H-pyridazin-3-ylideneamine; and
5-methyl-3-pyridin-3-ylmethyl-3H-[1,3,4]thiadiazol-2-ylideneamine
and pharmaceutically acceptable salts and optical isomers thereof.
